(19) United States
(12) Patent Application Publication
(10) Pub. No.: US 2022/0051773 A1
(43) Pub. Date: Feb. 17, 2022

(54) SYSTEMS, METHODS, AND APPARATUSES FOR MANAGING DATA FOR ARTIFICIAL INTELLIGENCE SOFTWARE AND MOBILE APPLICATIONS IN DIGITAL HEALTH THERAPEUTICS

(71) Applicant: BETTER THERAPEUTICS, INC., San Francisco, CA (US)

(72) Inventors: Kevin Appelbaum, Corte Madera, CA (US); Mark Berman, San Francisco, CA (US); Andres Camacho, Lafayette, CA (US)

(21) Appl. No.: 17/290,204

(22) PCT Filed: Oct. 31, 2019
(86) PCT No.: PCT/US2019/059259

§ 371 Date: Apr. 29, 2021

Related U.S. Application Data
(60) Provisional application No. 62/753,811, filed on Oct. 31, 2018.
(60) Provisional application No. 62/877,781, filed on Jul. 23, 2019.

(51) Int. Cl. G16H 20/10; G16H 10/40; G16H 10/60; G16H 20/70; G16H 50/20

(52) U.S. Cl. CPC G16H 20/10; G16H 10/40; G16H 50/20; G16H 20/70; G16H 10/60

(57) ABSTRACT

Disclosed herein are systems and methods of a digital therapy to identify and reinforce beneficial behaviors that are contributing to a patients progress toward achieving a desired health outcome, and to predictively identify an opportunity or need to adjust a patients medication.

### TECHNICAL FIELD OF DISCLOSURE

The subject matter disclosed herein generally relates to managing and providing a digital medication system for use in conjunction with a digital therapy, e.g. for the treatment of cardiometabolic disorders such as diabetes, and in particular to clinical decision support that can predictively determine whether a patient will reach a medication-adjustment threshold and alert the patient's clinician or other provider accordingly. Some of the aspects described herein for achieving this include mobile applications, machine-learning, and predictive analytics.

### BACKGROUND

Despite the long-standing, massive effort to develop effective methods for increasing healthy behavior in human subjects, the number of people worldwide who suffer from adverse cardiometabolic conditions such as obesity, cardiovascular disease, and metabolic disorders (e.g., type-II diabetes) is rapidly growing. These conditions result in numerous medical complications, a lowered quality of life, shortened lifespan, lost work productivity, a strain on medical systems, and a burden on medical insurance providers, all of which translates into increased costs for both individuals and society.

Indeed, Type-2 diabetes prevalence is at pandemic levels and continues to rise in the U.S. and globally. NCD Risk Factor Collaboration, The Lancet 387:1513-1530 (2016); Shi& Hu, The Lancet 383:1947-1948 (2014). Medication costs are rising in parallel and threaten to bankrupt national health systems. See, e.g., “National Diabetes Statistics Report 2017: Centers for Disease Control and Prevention, U.S. Department of Health and Human Services” (Internet); and “Economic Costs of Diabetes in the U.S. in 2012,” Diabetes Care 36(4):1033-1046 (2013). Despite increased use of medications and the advent of new pharmacological interventions, glycemic control among those with diabetes has not been improving since 2010. See Barnard et al., Change in Testing, Awareness of Hemoglobin A1c Result, and Glycemic Control in US Adults, 2007-2014 JAMA 318 (8):1825-1827 (2017).

A particularly vexing concern for diabetic patients in particular is the profound impact of clinical inertia, wherein health care providers fail to timely initiate, reduce and/or intensify pharmacological therapies due to a variety of triangulating factors. Reach et al., Clinical inertia and its impact on treatment intensification in people with type II diabetes mellitus, Diabetes & Metabolism 43:501-11 (2017). There may be gaps or otherwise inconsistent information in the data records for a patient, for example, making it difficult for the doctor to determine the efficacy of an ongoing course of treatment, let alone evaluate and/or predict future treatment adjustments—particularly before new test results or other biometric measurement data is received. Patient data may also be unavailable to the doctor for implementation reasons (e.g., doctor cannot access the data), or for practical reasons (e.g., patient non-compliance with regular diagnostic testing) and thus contributing to fewer data points than would/should otherwise be available. Clinical inertia may contribute to diabetic patients living with suboptimal glycemic control for many years, with dramatic consequences for their quality of life, morbidity and mortality, as well as the consequent huge costs for public health associated with uncontrolled diabetes.

Moreover, diabetes is viewed primarily as a chronic progressive disease, and accordingly there is a complete absence of evidence-based guidelines for reducing and/or eliminating pharmacologic intervention altogether in patients whose symptoms have improved. Indeed, doctors are not even trained in this respect, and thus any medication reductions that might be made are implemented in a completely ad hoc fashion. It would be truly remarkable for patients, providers and society if patients could be effectively weaned from some or all of their chronic medications in an informed and automated fashion when their condition improves.

Thus, there remains a need for technological solutions for, e.g., managing therapy regimens, such as lifestyle interventions, in a manner that provides improved compliance, improved outcomes, and/or long-term durability of improved health. In particular, there are myriad benefits that would result if clinician or other providers (e.g., doctors) could predictively foresee and adjust (e.g., reduce, increase) aspects of patient's treatment. Doctors typically adjust treatment based on tests and various measurements, either in real time (e.g., during a visit) or shortly thereafter. But there is no means for predictively determining ways to adjust treatment that could improve aspects of the treatment. What is needed then is a way to predictively adjust prescriptions during chronic therapy, even where there is a lack of all of the data points—particularly where data may be unavailable or imperceptible to a human.

### SUMMARY

Disclosed herein are systems and methods intended to address the above-described shortcomings in the art but may provide additional or alternative benefits as well. A digital behavioral therapy provider may create and publish a software application that provides healthcare therapeutic options as a supplement or an alternative to drugs, surgery, or other conventional treatments. Devices of the digital therapy provider may be used to automatically or manually generate therapy regimens that may address a health condition of a patient of the digital behavioral therapy, which may require the patient to perform various tasks and may instruct various devices to capture certain types of data related to the patient's therapy, including body metric measurements and information related to the number and quality of interactions between the user and aspects of the digital therapy, sometimes referred to as “user-generated” inputs. The digital therapy may calculate various metrics, such as scores and milestone determinations, to measure the patient's progress, and to predict the degree of treatment response. The scores can be determined using dynamically generated and updated scoring models.

In one aspect, the devices of the digital therapy provider may execute a variety of machine-learning algorithms and predictive analytics that can use historical and ongoing patient-specific data values to calculate a health score for a patient and to provide specific behavioral feedback based on same, with the intent of motivating a change in behavioral pattern(s) that may improve treatment outcomes. In some embodiments, the digital therapy can transmit information or alerts based on engagement-related data values that are theoretically modifiable (e.g., minutes of exercise; count, presence, or absence of skill-module(s) completed; or number of plant-based meals consumed) to motivate and/or reinforce changes. In some embodiments, the digital therapy can transmit information or alerts based on fixed data values (such as baseline biometric values) to provide context. In preferred embodiments, the devices of the digital therapy provider calculate a prioritized list of behavioral actions that are predictive of success in achieving a desired health score value or milestone, and transmits personalized feedback based on same to the patient's device and/or to their provider's device. The devices of the digital therapy provider may also continually adjust the predictive analytics models through any number of machine-learning techniques, as the digital therapy receives additional data values from the same patient, and/or for additional patients.

In another aspect, the devices of the digital therapy provider may execute a variety of machine-learning algorithms and predictive analytics that can use historical data to model the likelihood that a particular data value for a patient (e.g., blood sugar score or blood pressure) will change, the likely magnitude of such change, and/or whether that new data value will satisfy a medication-adjustment threshold in the future. In one embodiment, the predicted data value score can inform the current disease status for the patient, and/or to forecast their future disease status, even in the absence of consistent biometric data from the patient, and the digital therapy may transmit such information to the patient's clinician or other provider. In another embodiment, where the predicted data value score satisfies the medication-adjustment threshold, then the digital therapy may transmit an alert to a device of the patient's clinician or other provider, suggesting that the provider adjust the medication or other prescribed treatments at some point in the future. The devices of the digital therapy provider may also continually adjust the predictive analytics models through any number of machine-learning techniques, as the digital therapy receives additional data values for additional patients.

In one aspect, the invention provides computer-implemented methods for managing medication in a patient having a health disorder, comprising: a) providing to said patient a digital therapy for achieving one or more therapeutic milestones for said disorder; b) collecting subject-specific data values associated with a medication adjustment threshold for said disorder; c) determining by way of predictive analytics using said subject-specific data values whether a medication adjustment threshold has been or will be reached; and d) providing to the clinician or other provider for said patient a medication adjustment alert and/or recommendation if said threshold has been or will be reached within a treatment period. In preferred embodiments, the health disorder is a cardiometabolic disorder.

In another aspect, the invention provides computer-implemented methods for treating a patient having or at risk of having a cardiometabolic disorder, comprising: a) providing to said patient a digital therapy for achieving one or more therapeutic milestones for said cardiometabolic disorder; b) collecting subject-specific data values associated with a medication adjustment threshold for said cardiometabolic disorder; c) determining by way of predictive analytics using said subject-specific data values whether a medication adjustment threshold has been or will be reached; and d) providing to the coach, clinician or other provider for said patient a medication adjustment alert and/or recommendation if said threshold has been or will be reached within a treatment period.

In some embodiments, the cardiometabolic disorder is selected from diabetes, dyslipidemia, obesity, or hypertension. In one embodiment, the cardiometabolic disorder is diabetes, and the medication comprises one or more of sulfonylureas, meglitinides, biguanides, thiazolidinediones, or alpha-glucosidase inhibitors. In one embodiment, the cardiometabolic disorder is dyslipidemia and the medication comprise one or more of statins, bile acid binding resins, fibrates, niacin, omega-3 fatty acids, cholesterol absorption inhibitors. In one embodiment, the cardiometabolic disorder is hypertension and the medication comprise one or more of diuretics, beta-blockers, ace-inhibitors, angiotensin II receptor blockers, calcium channel blockers, alpha-2 receptor agonists, alpha-beta blockers, central agonists, adrenergic inhibitors, or vasodilators.

In exemplary embodiments, the subject-specific data values comprise biometric measurements (e.g., how often measured and what change from baseline), medication adherence, descriptive or demographic data (e.g., location, gender, email service used, consumer activities), service-interaction data (e.g., number of interactions with a coach, clinician, or other provider) and software-interaction data (e.g., actions or tasks performed, software features accessed by a patient, type of data captured from patient device, amount and/or frequency of interactions with software features), and the like. In preferred embodiments, the subject-specific data values comprise one or more engagement subject specific values and one or more biometric subject-specific values.

In some embodiments, the determining step comprises a multi-factorial weighted analysis and/or machine learning of two or more, three or more, four or more, five or more, six or more, seven or more, eight or more, or all of the subject-specific data values collected from the patient, performed by an operations server. In some embodiments, the treatment period is at least about one, two, three, four, five, six, seven, eight, nine, ten, eleven, twelve, thirteen, fourteen, fifteen or sixteen weeks.

In another aspect, systems for managing a therapy regimen are provided, the system comprising: an operations database comprising non-transitory machine-readable data configured to store one or more patient records; and an operations server comprising a processor configured to: a) generate a first algorithm for determining a therapy regimen for a patient based upon one or more data fields of a plurality of patient records in the operations database; b) generate a second algorithm for determining a likelihood of a value change based upon the one or more of the plurality of patient records; c) generate the therapy regimen for a patient based upon the one or more data fields of the patient record according to the first algorithm; d) determine the likelihood of the value change based upon the one or more data fields of the patient record according to the second algorithm; e) update a medication-value of the therapy regimen, in response to determining that the likelihood of the value change satisfies a medication-update threshold; and f) transmit, to a GUI of a provider device, at least one data field, the medication-value, and one or more fields of the therapy regimen.

In some embodiments, the operations server is further configured to: a) receive patient data from a patient device; b) store the patient data into the patient record of the patient; and c) transmit at least one data field to a GUI of the patient device.

In some embodiments, the processor is further configured to receive patient data from one or more devices over one or more networks, and store the patient data into the one or more data fields of the one or more patient records; and wherein the processor uses at least one data field of the patient data for the second algorithm.

In some embodiments, the operations database is further configured to generate the patient record in the operations database using the patient data, and wherein the patient data and the patient record are each associated with the patient.

In some embodiments, the processor is further configured to update the second algorithm based upon updated values of the one or more data fields received from at least one device over the one or more networks.

In some embodiments, the operations server is further configured to communicate patient data with an EMR server over one or more networks. In some embodiments, the operations server is further configured to communicate patient data with one or more consumer devices, each consumer device configured to generate patient data that is stored into the patient record of the operations database.

In another aspect, computer-implemented methods for predicting a health outcome in a patient having a health disorder are provided, comprising: a) providing to said patient a digital therapy for achieving one or more therapeutic milestones for said disorder; b) collecting subject-specific data values associated with said one or more therapeutic milestones for said disorder; c) calculating a health score by way of a classifier system, wherein the classifier system comprises a machine-learning trained biomarker model and the classifier uses said subject-specific data values as input and determines a health score indicating whether a health outcome threshold has been or will be reached as output; and d) calculating and assigning an importance value for one or more, or all, subject specific data values, for the biomarker model.

In some embodiments, the computer-implemented method further comprises step e) transmitting to the patient and/or their clinician or other provider one or more of the health score, behavioral actions that are predictive of success in achieving the health outcome, and clinical alerts. In some embodiments, the health score is recalculated with every new engagement entered in the digital therapy. In some embodiments, the one or more behavioral actions are associated with one or more, or all, subject-specific data values rank ordered by the calculated importance value.

In one exemplary embodiment, the health disorder is a cardiometabolic disorder. In another exemplary embodiment, the health disorder is hypertension. In another exemplary embodiment, the health disorder is dyslipidemia.

In some embodiments, the biomarker model is a machine learning model, preferably a tree ensemble method, more preferably a random forest model. In some embodiments, the biomarker model is trained on one or more engagement and/or biometric subject-specific data values. Preferably, the biomarker model is trained on both engagement subject-specific data values and biometric subject-specific data values. In exemplary embodiments, the biomarker model is trained on one or more, or all, of the following subject-specific data values: counts of actions related to the use of the digital therapeutic, count of all meals reported, plant-based meals reported, physical activity reported, length of exposure to the intervention, baseline systolic, baseline diastolic, mean systolic and diastolic at training window end, initial systolic and diastolic change (end training mean—baseline), minutes of physical activity, and baseline Body Mass Index (BMI). Remarkably, the biomarker models of the present invention can be predictive even in the absence of continuous and/or updated biometric data from a given patient.

In some embodiments, the method comprises calculating the importance value by using gradient values provided by the classifier. In some embodiments, the method comprises calculating the importance value by accessing information about nodes in a tree model of the classifier. In preferred embodiments, the method comprises calculating the importance value by generating a Tree Shaply Additive Explanation value or by performing a regression analysis. In some embodiments, calculating and assigning the importance value comprises transmitting at least one score request to the classifier to generate the importance value and receiving the importance value from the classifier as a response to each score request.

In another aspect, computer-implemented methods of improving a health outcome in a patient suffering from, or at risk of, a cardiometabolic disorder are provided, comprising: a) providing to said patient a digital therapy for achieving one or more therapeutic milestones for said disorder; b) collecting subject-specific data values associated with said one or more therapeutic milestones for said disorder, including at least one baseline physiologic parameter associated with said health disorder; c) applying each of the one or more subject-specific data values against a trained classifier, wherein the classifier has been trained with a statistically significant plurality of subject-specific data values associated with one or more of a plurality of subjects, at least some of the plurality of subjects having the cardiometabolic disorder, and d) based on the applying, calculating a predicted change in the at least one physiological parameter of the patient. In one embodiment the method further comprises step e) transmitting the predicted change in the at least one physiological parameter of the patient to the patient and/or to a patient's clinician or other provider.

In some embodiments, the cardiometabolic disorder is selected from diabetes, dyslipidemia, obesity, or hypertension. In one embodiment, the cardiometabolic disorder is diabetes and the at least one physiological parameter is HbA1c level. In one embodiment, the method further comprises step e) transmitting a predicted change in HbA1c level to the patient and/or to a patient's clinician or other provider. In exemplary embodiments, the patient is being treated with one or more of sulfonylureas, meglitinides, biguanides, thiazolidinediones, or alpha-glucosidase inhibitors and/or at least some of the plurality of subjects were undergoing treatment with one or more of sulfonylureas, meglitinides, biguanides, thiazolidinediones, or alpha-glucosidase inhibitors.

In one embodiment, the cardiometabolic disorder is dyslipidemia and at least one physiological parameter is cholesterol level, LDL, and/or HDL. In one embodiment, the method further comprises step e) transmitting the predicted change in cholesterol level, LDL, and/or HDL to the patient and/or to a patient's clinician or other provider. In exemplary embodiments, the patient is being treated with one or more of statins, bile acid binding resins, fibrates, niacin, omega-3 fatty acids, cholesterol absorption inhibitors and/or at least some of the plurality of subjects were undergoing treatment with one or more of statins, bile acid binding resins, fibrates, niacin, omega-3 fatty acids, cholesterol absorption inhibitors.

In one embodiment, the cardiometabolic disorder is hypertension and the at least one physiological parameter is blood pressure. In one embodiment, the method further comprises step e) transmitting the predicted change in blood pressure to the patient and/or to a patient's clinician. In exemplary embodiments, the patient is being treated with one or more of diuretics, beta-blockers, ace-inhibitors, angiotensin II receptor blockers, calcium channel blockers, alpha-2 receptor agonists, alpha-beta blockers, central agonists, adrenergic inhibitors, or vasodilators and/or at least some of the plurality of subjects were undergoing treatment with one or more diuretics, beta-blockers, ace-inhibitors, angiotensin II receptor blockers, calcium channel blockers, alpha-2 receptor agonists, alpha-beta blockers, central agonists, adrenergic inhibitors, or vasodilators.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the relevant art to make and use the disclosure.

FIG. 1 shows components of a system, according to an exemplary embodiment.

FIG. 2 shows a provider-GUI displayed on a provider device, according to an exemplary embodiment.

FIG. 3 shows Receiver Operator Characteristics (ROC) curves for machine learning model predicting systolic change (SC) and a model predicting systolic change without use of ongoing blood pressure data (SC-APP).

FIG. 4 illustrates how SHAP values can be used to show how explanatory variables contribute to success in meeting a response variable (e.g., improvement in systolic blood pressure ≥10 mmHg). In this figure, the feature list down the y-axis is in order of contribution to the model (most to least). Each dot represents the value for one participant. SBP change and DBP change are the difference in measurements from baseline to the end of the 28-day training period.

FIG. 5 shows SHAP values for explanatory variables for 2 participants. The SHAP value plotted on the y-axis indicates that amount the variable positively or negatively contributes to the prediction of success (the output value). The probability threshold (output value that assigns a prediction of success) used in this example is 0.66.

### DETAILED DESCRIPTION

Certain illustrative aspects of the systems, apparatuses and methods according to the present invention are described herein in connection with the following description and the accompanying figures. These aspects are indicative, however, of but a few of the various ways in which the principles of the invention may be employed and the present invention is intended to include all such aspects and their equivalents. Other advantages and novel features of the invention may become apparent from the following detailed description when considered in conjunction with the figures.

In the following detailed description, numerous specific details are set forth in order to provide a thorough understanding of the invention. In other instances, well known structures, interfaces and processes have not been shown in detail in order not to unnecessarily obscure the invention. However, it will be apparent to one of ordinary skill in the art that those specific details disclosed herein need not be used to practice the invention and do not represent a limitation on the scope of the invention, except as recited in the claims. It is intended that no part of this specification be construed to effect a disavowal of any part of the full scope of the invention. Although certain embodiments of the present disclosure are described, these embodiments likewise are not intended to limit the full scope of the invention.

Digital Medication Systems and Software Applications

A digital therapy provider may host a digital therapy and may create and publish a therapeutic software application that provides healthcare therapeutic options as a supplement or an alternative to drugs, surgery, or other conventional medical treatments. Patients of the digital therapy provider may install the therapeutic software application onto a patient device (e.g., mobile device, laptop computer, workstation computer, server), and then the patient may use the patient device to load, execute, and access the various features of the software application. When initially registering with the digital therapy or otherwise beginning to use the application, a patient may indicate a particular health condition (e.g., diabetes, dyslipidemia, high blood-pressure) to be addressed and may then provide certain initial data inputs, which the digital therapy may use to prepare an appropriate therapy regimen to address the indicated condition. The patient may then access the features of the software application to begin tasks assigned to them by the therapy regimen, such as a diet or exercise routine, among other possible tasks. The software application may provide patients with various graphical user interfaces (GUIs) that allow the patient to, for example, interact with the features of the software application in a human-friendly way, submit data inputs, log journal entries of the patient's progress, and receive information and/or feedback related to their treatment and therapy regimen.

Unlike conventional consumer-facing health, diet, fitness, and lifestyle software applications, sometimes called “apps” when installed and executed on a mobile device, the features of the software application of the digital therapy described herein are a medical prescription, as opposed to general “well-being” tracking. Conventional well-being software products are essentially “voluntary” products that are premised on a user's voluntary participation and cue off the user's selected goals, so the functionality is broadly open to user changes and at-will interactions, but not premised on adherence to a particular regimen or other set of tasks. As the digital therapy software described herein is subject to a medical prescription, a patient's therapy regimen is determined for a patient by the digital therapy provider, algorithmically and/or according to inputs from personnel of the digital therapy provider, with little or no input from the patient. Likewise, because activities (e.g., meals, exercise, journal entry) assigned to the patient according to the therapy regimen are “dosages” that are part of a medical prescription, rather than a generic well-being improvement effort, the patient must adhere to the therapy regimen. The software described herein may comprise various features and functions that facilitate patient adherence and track patient adherence to a therapy regimen, in a way that conventional software applications do not.

Embodiments of the systems and methods disclosed herein may include one or more computing devices, such as an operations server or any other computing device, that may calculate a health score for each patient. In many instances, a health score may be a representation of a patient's progress, the status of the patient's therapy regimen, and/or a likelihood of success. A computing device may execute one or more artificial intelligence and/or machine learning software programs to generate and update health score modeling algorithms, and/or generate and update the health scores of patients. One having skill in the art would recognize that the artificial intelligence and machine learning software may execute various artificial intelligence and machine learning algorithms and processes, such as generalized linear models, Kalman filter models, Gaussian processes, tree-ensemble methods (e.g., random forests, gradient boosting trees), support vector machines, unsupervised and/or supervised clustering, and deep learning (e.g., neural networks), among others. In some examples, machine learning software may “learn” (e.g., update data processing algorithms according to historical data trends) from training data, which, in some instances, includes labeled data. An operations server may apply a health score model to calculate a health score, where the health score model may indicate parameters (e.g., data fields) and/or relative-weights for the parameters when calculating a corresponding health score. The health score and the corresponding health score model may be a function, at least in part, of the interactions between the patient and aspects of the digital therapy (e.g., software application, coach). As described herein, the health score is based on a health condition that the patient wishes to address using the features and functions provided by computing devices of the digital therapy and the patient's device. As described herein, the health score is calculated by an operations server or other computing device of the digital therapy using health score models that accept parameters that are relevant to the patient's health condition, where the parameters correspond to expected types of data stored in certain data fields. In one embodiment, the health score of a patient addressing diabetes may be calculated by the operations server using a health score model that is trained with and uses data fields relevant to monitoring diabetes, such as blood glucose level and weight. In another embodiment, the health score of a patient addressing hypertension may be calculated by the operations server using a health score model that is trained with and uses data fields relevant to monitoring hypertension, such as systolic and diastolic blood pressure.

However, it should be appreciated that health scores may be tailored to calculate values representing broader or narrower qualities of a patient. As an example, a health score may represent an “overall” health status of a patient. In this example, the corresponding health score model may be trained with and use a plurality of data fields, such that the operations server or other computing device applying a health score model outputs a calculated value representing a broad understanding of the patient's health. As another example, the health score may more narrowly represent the patient's progress in addressing diabetes with respect to middle-aged males. In this example, the health score is more narrowly tailored to the patient. Because the health scores and the corresponding health score models may vary, it could also be appreciated that the operations server or other computing devices of the digital therapy may calculate any number of health scores for patients of the digital therapy provider. For instance, a patient may receive a health score that is relative to their progress and/or therapy regimen status in addressing a health condition, and a health score that represents their overall health. The health score, in many implementations, may be determined by an operations server or other computing device using baseline body metric measurements and a series of updated data values received from, for example, a patient device at certain times over the course of treatment. In this way, the health score may be based, in part, on the patient's personal progress and/or the status of their therapy regimen.

A health score model applied by an operations server or other computing device of the digital therapy may be trained and re-trained using any number of known statistical and regression algorithms against a predetermined set of data fields (e.g., blood glucose, cholesterol, blood pressure, weight, number of interactions with the software or coach). Any number of supervised learning techniques can be used to train a health score model. For example, in some cases a random forest regression can be trained on historical data for diabetes patients to predict A1C loss, where the historical data may be, for example, database records for individuals who have a diabetes health condition as indicated by one or more data fields of the database records. In this diabetes-related exemplary health score model, a target variable, A1C values or loss, is continuous. In another example, a gradient boosted classifier can be trained on historical database records for individuals, to determine a likelihood for whether or not a patient is going to lose over 5% of a body weight value in 12 weeks. In this exemplary model, the target variable may be a binary value (e.g., 1 or 0).

The operations server, or other device of the digital therapy, may retrain health score models at given time intervals or when certain events occur. For instance, the operations server may retrain a health score model for a condition each time a threshold number of patients complete a therapy regimen addressing the condition. In some cases, health score models may be tailored to be applied only at a certain moment in a patient's therapy timeline. For example, a patient may receive a 12-week therapy regimen to address high blood-pressure, where the patient's health score is calculated and updated each week. In this example, the health score after the third week is calculated using a health score model that was trained using the data records of other patients treated for high blood-pressure after three weeks. A different health score model is then applied after each successive week. In such cases that use timeline-based health score models, the operations server may retrain a health score model after being used for calculations a threshold number of times.

The operations server may retrain the health score models either with or without input from personnel of the digital therapy provider. In an automated implementation, the operations server may retrieve values of certain data fields from patient records that are stored in a patient database, and may then reapply the particular training algorithm. In some cases, the values of the data fields may include health scores calculated for the patients of the patient records and/or the number of interactions between such patients and the software or a coach. In implementations involving personnel of the digital therapy provider, an administrator or other person, may review the data records and manually retrain the health score model via a graphical user interface (GUI) by, for example, manually adjusting algorithms used to train a health score model and/or adjust the algorithm that is actually used by the health score model to calculate a health score.

In addition or as an alternative to sophisticated health score calculations and handling health score models, an operations server or other computing device of the digital therapy may compare one or more data fields relevant to the patient's condition, against pre-stored milestone parameters or data values. For example, a database record for a patient addressing diabetes may have data fields for blood glucose levels and weight, among others. At certain predetermined milestone intervals, the operations server may compare the patient's recent blood glucose level against a pre-stored blood glucose level. The pre-stored milestone values may operate as threshold values that may trigger certain functions in the operations server or other devices, such as generating a notification interface to be presented at the GUI of a coach computer when a patient's data value fails to satisfy the corresponding pre-stored milestone value. In some implementations, there may be multiple pre-stored milestone values for multiple data fields, which may cause the operations server to compare multiple different patient values of different fields against multiple different pre-stored values. Additionally or alternatively, in some implementations, there may be multiple pre-stored values for a data field, such that the pre-stored values function as, for example, upper-bound and lower-bound thresholds, thereby facilitating more potential responses from the operations server.

A health score value may be applied by the digital therapy in a number of ways. The health score value may be used to determine whether the patient is compliant with their assigned treatment. Patients may be required to conduct regular meetings with a coach to discuss their treatment. The health score value may be useful to prepare for and conduct the meeting. It is also useful for determining how the treatment is progressing generally. As such, the operations server may retrieve the health score value from the patient's data records and transmit it to a patient device or coach computer, where the health score may be presented to the patient or the coach on one or more GUI displays. In addition, a GUI may display a patient's health score value with other health score values, such as an average health score value or other patients' health score values. In this way, the patient or coach may understand the patient's health score with wider context.

A health score or milestone determination may be used to vary a patient's therapy regimen. For example, a patient whose weight fails an upper bound threshold may receive updated diet instructions. In operation, after the operations server determines the weight value of the patient's data record fails a pre-stored value for weight, one or more data fields pertaining to the patient's diet instructions may be updated to include foods having less fat or sugar. In some cases, determining whether the data values satisfy one or more pre-stored values may be indicators of the patient's progress through their therapy regimen and/or an indicator of the status of the therapy regimen.

As a component of determining a patient's milestones, calculating scores, and/or monitoring their progress generally, the operations server may receive indicators of interactions between the patient and aspects of the digital therapy (e.g., coach, therapeutic software application). The indicators of interactions may be received by operations server as data received from various devices (e.g., patient device, coach device, data contribution device). The operations server may use these indicators to track an amount of interactions between the user and various aspects of the digital therapy. In some circumstances, the therapy regimen of a patient requires the patient to have a certain amount of interactions with a coach and the therapeutic software application, and the indicators of interactions may allow the operations server or a coach to review the patient's engagement and involvement with their therapy regimen. Non-limiting examples of indicators of interactions may include whether a meal plan was followed; whether a therapy regimen task was completed; whether a coaching call was completed; whether one or more, or all, ingredients on a shopping list were procured; whether the subject consumed a specified amount of water or whether the subject consumed water; a number of meal plan meals consumed, a number of tasks completed, a total number of calories burned in one or more activities, a number of coaching calls completed, length of one or more coaching calls, a number or fraction of ingredients procured from a shopping list, a number of hydration events, or a total volume of hydration.

The health score model may also be applied by an operations server or other computing device of the digital therapy to provide personalized behavioral feedback to each patient based on analysis of the data values contributing to that patient's health score, to identify behaviors increasing their likelihood of success in achieving a desired health outcome. Modifiable data values such as engagement data (e.g. meals consumed, skill module(s) completed, or minutes of exercise) can be highlighted to motivate and/or reinforce behavioral changes, while non-modifiable data values such as historical and/or current biometric data can be displayed to add context. Analytical techniques analogous to coefficient analysis in classical regression can be used to identify and prioritize the contributing data values. For example, in some cases a Tree Shapley Additive Explanation (SHAP) algorithm is applied to the random forest regression to attribute an importance value for each data value by the operations server, and behaviors associated with one or more of the prioritized data values can be transmitted to a patient device or coach computer and presented to the patient or the coach on one or more GUI display alerts.

Clinical Decision Support Software

The digital therapy may publish clinical decision support (CDS) software that clinician or other providers (e.g., doctors, pharmacists) may operate after a doctor has prescribed the digital therapy to a patient. The doctor may operate the CDS software to interact with patient treatments after the patient's therapy regimen has been generated. The operations server may execute one or more algorithms that support various features and functions of the CDS. In some implementations, the operations server may execute algorithms for predictive treatment functions underlying the CDS software. Conventional software algorithms could potentially receive data inputs at a server, which in turn outputs some value that could then be provided to a user. Regulatory agencies, such as the US Food and Drug Administration (FDA), are responsible for regulating and validating prescription drugs and medical devices before they are prescribed by clinicians or other providers (e.g., doctors, pharmacists). Moreover, doctors are regulated by a variety of sources, schools-of-thought, and principles, governing patient treatments. Consequently, there are a variety of reasons why the basic algorithmic approach of conventional software will not be sufficient for an adequate and safe medically-prescribed digital software. In such embodiments, the operations server may receive inputs from a variety of data sources, which may include data captured by, e.g., various consumer devices, inputted by users on an end-user device, and third-party servers. The operations server may use the data to generate predictive adjustments to a patient's treatment. In some cases, the operations server may transmit an alert to a doctor's device suggesting the doctor adjust the treatment.

Components of an Exemplary System Architecture

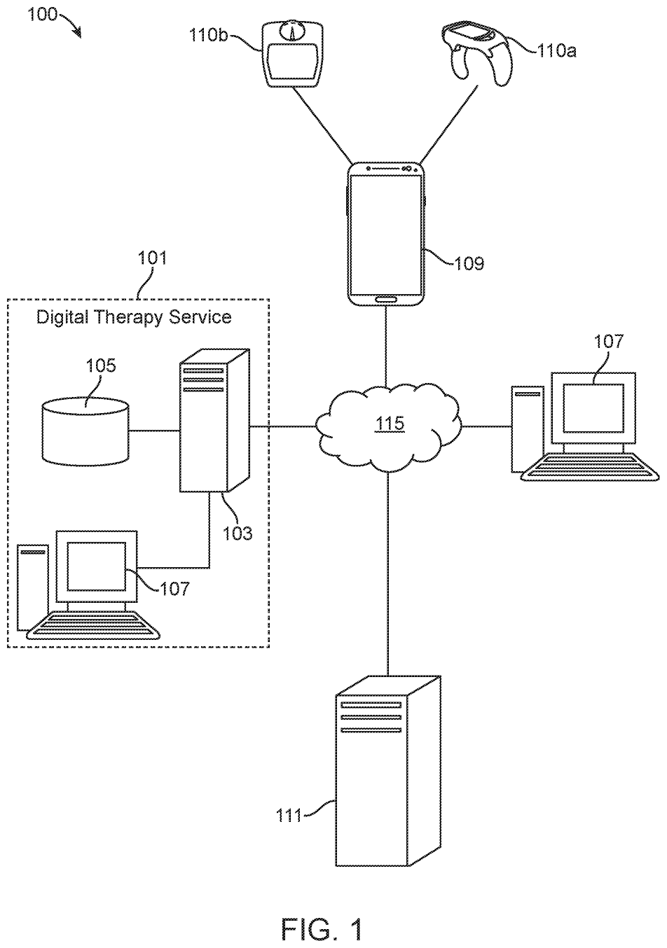

FIG. 1 shows components of a system 100, according to an exemplary embodiment. The exemplary system 100 may comprise a digital therapy 101 of a digital therapy provider, a patient device 109 (sometimes referred to as “mobile devices” or “user devices” herein), one or more consumer devices 110, an EMR server 111, a provider device 113. The digital therapy 101 may comprise various computing devices (e.g., servers, databases) that are accessible to external devices via one or more networks 115 (e.g., Internet, intranet, extranet, VPN). In the exemplary system 100, the various computing devices of the digital therapy 101 may include, for example, an operations server 103, an operations database 105, and a coach device 107. The digital therapy 101 may further include one or more internal service-networks (not shown) connecting the devices of the digital therapy 101. It should be appreciated, however, that a digital therapy 101 and/or system 100 may comprise any number of computing devices (e.g., servers, workstations, laptops) and networking devices (e.g., routers, switches, firewalls) configured to perform various processes and tasks described herein. It should be further appreciated that the exemplary system 100 shown in FIG. 1 is not limiting on the variety possible embodiments that may have additional, alternative, or omitted features from the features shown in the exemplary system 100.

For ease of explanation, only one of each device is shown in FIG. 1. However, one having skill in the art would appreciate that some embodiments may comprise any number of devices, which may function in a distributed-computing environment for redundancy and/or load-bearing purposes. It should also be appreciated that such devices may be embodied on the same device or split among many devices, such that a single computing device may perform the functions described herein as being performed by multiple devices, or such that multiple computing devices may perform the functions described herein as being performed by a single device. For example, in some embodiments, certain processes and features described herein as being executed and provided by an operations server 103 may be distributed among and executed by a number of computing devices, which consequently execute such processes and provide such features of the operations server 103.

Operations Server 103

An operations server 103 of the digital therapy 101 may generate, manage, and update data associated with patients and patient therapy regimens. The operations server 103 may be any computing device comprising a processor and machine-readable memory capable of performing various processes described herein. Non-limiting examples of an operations server 103 may include a workstation computer, a laptop, server, mobile device (e.g., smartphone, tablet), and the like. As described herein, the operations server 103 may perform any number of processes driving the technical features of the digital therapy 101, and generally providing patients therapeutic services. However, it should be appreciated that the features of the operations server 103 may be performed by, or otherwise distributed among, any number of computing devices.

The operations server 103 may execute various processes that allow the operations server 103 to function as an entryway to the features of the digital therapy 101. The operations server 103 may receive inputs from a patient device 109, consumer devices 110, an EMR server 111, and a provider device 113. The operations server 103 may also transmit to the various data files and/or executable instructions to devices of the system 100 (e.g., coach device 107, patient device 109, provider device 113, EMR server 111). For instance, a patient device 109 may, for example, execute therapeutic software published by the digital therapy provider that develops, owns, builds, and/or operates the digital therapy 101. The operations server 103 may be accessed by patients through the therapeutic software on the patient device 109, which allows patients to interact with the various services and features provided by the various software and hardware components of the digital therapy 101. Likewise, a provider device 113 may, for example, execute therapeutic software directed to clinicians or other providers (e.g., doctors, pharmacists) published by the digital therapy provider. The operations server 103 may be accessed by a doctor through the provider software on the provider device 113, which allows the doctor to interact with the various services and features provided by the various software and hardware components of the digital therapy 101.

In some implementations, the operations server 103 may generate, manage, and update patient health and treatment related data about a particular patient and collection of patients, where the data records may be stored into an operations database 105. The operations server 103 may manage patient data based on data inputs received from various devices of the system 100 (e.g., coach device 107, patient device 109, consumer devices 110, provider device 113, EMR server 111). In some implementations, the operations server 103 may communicate with an EMR server 111, allowing the operations server 103 to receive and/or transmit patient data records stored with a third-party EMR service (e.g., insurance company).

In some implementations, an operations server 103 may generate a therapy regimen to treat a particular condition of a patient. The patient's condition may be indicated, for example, through a graphical user interface (GUI) by a patient operating a patient device 109, a coach operating a coach device 107, or a clinician or other provider operating a provider device 113. The operations server 103 may receive data inputs associated with treating the patient's condition or therapy regimen, sometimes referred to herein as “parameters,” from various data sources (e.g., coach device 107, patient device 109, consumer device 110, or provider device 113). The operations server 103 may use the incoming data to calculate various scores, such as a health score, to indicate a patient's likelihood of success and/or progress toward improving their health with respect to the condition or overall health.

The therapy regimen may be a collection of machine-readable data and executable code that inform operation of a therapeutic software applications published by the digital therapy 101 and executed by a patient device 109 and a provider device 113. For instance, the software code of the therapeutic application of a patient device 109 may be configured to generate one or more interactive GUIs, according to instructions of the therapy regimen; each GUI may be displayed to the patient operating the patient device 109. The operations server 103 may generate tasks associated with the therapy regimen for patients and coaches to perform, such as diet routines, exercise routines, and coaching sessions. Details regarding the tasks may be presented to patients, coaches, and providers via the interactive GUIs; and the patients, coaches, and providers may input data as well.

The operations server 103 may also execute any number of automated processes according to certain executable code as part of the therapy regimen. For example, code associated with a therapy regimen addressing a diabetes condition may, at a predetermined time, instruct the operations server 103 to query blood glucose data of a patient in an operations database 105 and then compare that blood glucose data against pre-stored blood glucose stored data in, e.g., an operations database 105.

The operations server 103 may execute software configured to support CDS software for a clinician or other provider (e.g. doctor, pharmacist). In some cases, the CDS software may be locally installed on the provider device 113. In some cases, the operations server 103 may comprise webserver software (e.g., Apache, Microsoft IIS) hosting a website having a “web portal,” “web app,” or “cloud server.” The provider device 113 may access the website via one or more networks 115. In operation, the operations server 103 may receive data inputs and execute one or more algorithms configured for predictive analysis and machine-learning. The software-based predictive analytics algorithm may reference any number of data fields of patient records stored in an operations database 105 when predicting whether to suggest adjustments to a patient's therapy regimen.

Often, however, there may be “gaps” or otherwise inconsistent information in the data records for a patient. This would make it difficult for a doctor to determine the efficacy of an ongoing course of treatment, let alone predict future treatment adjustments—particularly before new test results or other biometric measurement data is received. It is also notable that the data on a patient may include data that is unavailable to a doctor for implementation reasons (e.g., doctor cannot access the data) and/or practical reasons (e.g., imperceptible to a human). As an example, a patient who has diabetes may visit their doctor who is operating a provider device 113 having CDS software capable of receiving alerts from the operations server 103; where the alerts may indicate a suggestion for changing the patient's treatment. The CDS software in the exemplary system 100 may be instructed to deliver an alert to a provider device 113 when a patient's blood sugar is likely to drop below a predetermined threshold, and thus predict that the patient's medication should be decreased. In some conventional approaches, when an inputted blood sugar value has been received by the operations server 103 from a device of the system 100, the operations server 103 may subsequently determine that the blood sugar value has failed to satisfy some predetermined threshold value. But improving upon those conventional technologies, the operations server 103 may further generate and deliver alerts based on, for example, an incomplete set of actual values and/or value changes. The operations server 103 may also generate and deliver alerts when a doctor does not or cannot know all of the information. The predictive analytics resolve these issues because the algorithms are dynamically generated through machine-learning algorithms that are based on a large swath of data values. Non-limiting examples of the data values employed by the predictive analytics may include biometric measurements, descriptive or demographic data (e.g., location, gender, email service used, consumer activities), service-interaction data (e.g., number of interactions with a coach) and software-interaction data (e.g., actions or tasks performed, software features accessed by a patient, type of data captured from patient device 109, amount of interactions with software features), among others.

As an example, the operations server 103 may execute predictive analytics that are based, at least in part, on blood sugar level, patient vital data (e.g., age, weight, gender, location), and indicators for the number of times that a patient accessed certain features. In this example, a patient may be regularly engaged with the various features of the therapy software executed on the patient device 109, but is failing to log their blood sugar, thereby making it impossible for a doctor to determine whether that patient's treatment should be adjusted; particularly lowering the patient's medication. But that is not the case for the predictive analytics of the operations server 103. Rather, the operations server 103 may generate a blood sugar score-probability indicating whether the patient's blood sugar is changing and going to satisfy a blood sugar score threshold in the future. As another example, the patient may be logging their blood sugar, but not with enough regularity that a doctor and/or conventional software analysis could determine whether to lower medication based solely on the blood sugar values. For instance, at some moment during the course of treatment, the data records may have only two blood sugar values, whereas the data records should have more (e.g., 40 or 50) blood sugar value entries for the patient. This shortage should ordinarily constitute too few data points to make a definitive determination for, e.g., a blood sugar score, a probability of whether the blood sugar level will come down, and a determination that the medication should be adjusted. That is, a human or simplistic test against thresholds would ordinarily be unable to make a determination whether to prospectively adjust the patient's medication or treatment, because conventional means merely consider past results. However, the predictive analytics may use a variety of other markers and data fields (such as the patient's geographical region, age, and gender, among others) to predict, e.g., the patent's future blood sugar score, allowing the operations server 103 to determine whether it is likely the patient's blood sugar score will satisfy a threshold. As such, the operations server 103 can predictively determine that the patient's medication may be adjusted, even without having all of the detailed records that would ordinarily be required. The operations server 103 may then generate an alert, which may be transmitted and displayed on a GUI of the provider device 113.

In operation, the operations server 103 may comprise software code that may output a prediction of likelihood for a particular value change or absolute value, based on the pre-stored historical data from a body of patients' data records. The operations server 103 may determine the likelihood of success as a function of, for example, engagement with the digital interface (e.g., number of interactions between a patient and the therapeutic software application), engagement with the lifestyle regimen (e.g., rate of compliance with therapy regimen activities, meal plans, and the like). The likelihood may also be determined as a function of one or more parameters. For example, the likelihood can be determined as a function of whether a meal plan was followed or logged, or, if not, whether an alternative meal was consumed or logged; whether a lifestyle regimen activity was completed or logged; whether a coaching call was completed or logged; whether ingredients on a lifestyle regimen shopping list were procured or logged; whether the patient consumed a specified amount of water or whether water-intake was logged; a number of meal plan meals consumed, a number of activities completed, a total number of calories burned in one or more activities, a number of coaching calls completed, length of one or more coaching calls, a number or fraction of ingredients procured from a shopping list, a number of hydration events, a total volume of hydration logged, or a number or rate of body weight measurements performed. In some cases, the likelihood can be determined as a function of type or amount of, for example, physical activity completed and/or logged.

In some cases, the likelihood of a future score is determined by a multi-factorial weighted analysis of two or more, three or more, four or more, five or more, six or more, seven or more, eight or more, or all types, amounts, or rates that inputs for certain patient-performed tasks are received (e.g., engagement with interface, engagement with therapy regimen, parameters, and/or type of, for example, physical activity completed). The weights and task types can be identified manually via a GUI or computationally via, for example, logistic regression or machine learning, methods to identify patterns associated with a high or low likelihood of therapeutic milestone achievement. For example, if a patient enters a low number of patient-related inputs compared to a predetermined relative threshold (e.g., relative to other members of a patient cohort, or relative to prior patients in the database, or a portion thereof) into a GUI of the patient device 109 the comparison engine of the operations server 103 may determine that the patient is unlikely to achieve a lifestyle-related health condition milestone. As another example, if a patient enters a high number of patient-related inputs into a GUI of the patient device 109 compared to a predetermined relative threshold, the comparison engine of the operations server 103 may determine that the patient is likely to achieve a particular value score for a particular data field that will satisfy a medication-adjustment threshold.

The operations server 103 may continuously update the predictive algorithm at a predetermined interval or in real-time, as additional data fields are added to the body of patient records. This allows the operations server 103 to autonomously evolve the predictive analytics scoring precision.

In some embodiments, users (e.g., patients, providers, coaches) may access the operations server 103 through a web-based interface, in addition or as an alternative to accessing the operations server 103 through locally installed software. In such embodiments, the operations server 103, or other computing device of the digital therapy 101, may execute webserver software (e.g., Apache®, Microsoft IIS®), which may provide remote devices (e.g., coach device 107, patient device 109, provider device 113) access to the features and processes hosted and executed by the operations server 103, and, more generally, may host and serve features of the digital therapy 101 using typical web-technology protocols (e.g., TCP/IP, HTTP). In some implementations, software applications executed by the devices of the system 100 may provide data for GUIs generated by native-software interfaces, such that a user does not require web browser software to remotely access the features of the digital therapy 101.

The operations server 103 may perform a number of additional features and processes, many of which are further described herein. These additional processes may include, but are not limited to: tracking a patient's progress through their therapy; generating health score models; calculating health scores; identifying milestone achievements; identifying and reinforcing beneficial behaviors based on a prioritized list of contributing parameters, and managing access to various features of the digital therapy 101 through user login procedures. It should be appreciated that this listing is merely exemplary and is not intended to be exhaustive.

Operations Database 105

The digital therapy 101 may comprise one or more databases that store data records containing data related to patients and software execution, represented in FIG. 1 as an operations database 105. An operations database 105 may be hosted on any computing device comprising non-transitory machine-readable storage and a processor capable of querying, retrieving, and updating data records of the database. In operation, computing devices of the system 100, such as an operations server 103 or a provider device 113, may query and/or update the operations database 105 via one or more networks 115. In some embodiments, the operations database 105 may be a single device hosting the database; and, in some embodiments, the contents of the operations database 105 may be distributed among a plurality of computing devices.

The operations database 105 may store data records containing data used for application operation, where the fields of such data records contain various types of data related to the processes executed by the various devices of the system 100. The operations database 105 may contain data records used for selecting, generating, and/or providing therapy regimens for devices of the system 100 to access via an application designed for a user (e.g., patient, doctor) and an associated device (e.g., coach device 107, provider device 113).

In some implementations, the operations database 105 may contain data records used for generating and updating health scores and other forms of data for patients. The operations database 105 may store health score models that the operations server 103 may access to calculate, e.g., a patient's health score or when the operations server 103 is instructed to retrain the health score model. Similarly, the operations database 105 may contain data for determining whether to predictively adjust the patient's treatment or medication, which the operations server 103 may access when executing the predictive analytics software routines.

In some embodiments, an operations database 105 may store patient profile data records containing data fields for various types of data that describes a patient or related to patient therapy. Non-limiting examples of information stored in patient profile database records may include, for example, basic information about the patient (e.g., name, date of birth, occupation, email service, geographic location), a patient identifier (“patient ID”), health-related data (e.g., body metric measurement data, a condition, therapeutic goal), treatment-related data (e.g., calculated scores, text of journal entries, text of coach interactions, patient-application interactions), and the like. In operation, the operations database 105 may respond to query and update requests that are received from an operations server 103 or other devices of the system 100.

End-User Devices

Coach Device 107

The digital therapy 101 may comprise any number of service provider devices; an exemplary use of a service provider device may be, e.g., a coach device 107. A coach device 107 may be any computing device comprising a processor and machine-readable memory capable of executing the various processes described herein. Non-limiting examples of the coach device 107 may be a workstation computer, a laptop computer, a server, a mobile device (e.g., smartphone, tablet), and the like. The coach device 107 may be configured for use and operation by a coach. As mentioned, although only coach device 107 is shown as an exemplary service provider device in FIG. 1, some embodiments may comprise multiple service provider devices configured with the functions and configurations needed by the various other personnel of the digital therapy 101.

The coach device 107 may have one or more GUIs that allow a coach to interact with coach software and provide instructions to the coach device 107, providing the coach with access to, for example, patient profiles and various other devices and features of the system 100. In some implementations, the coach may use the coach device 107 to generate various data inputs for the operations server 103 to consume for particular processes or analyses, sometimes referred to as a “coach input.” The coach device 107 may also query and manage data about patients stored in an operations database 105. As an example, using a coach device 107, a coach may submit a query to the operations database 105 to retrieve and/or update information about a particular patient. The data records of the patient may be organized by patient IDs, and thus the appropriate data record may be retrieved according to the patient ID identified in the query.

A GUI of the coach device 107 may be organized as a dashboard or similar presentation that allows a coach to review the data of one or more patients under the coach's care. The software of the coach device 107 may query the data of the patient's data record, and present the data in an organized GUI. The coach may use this dashboard GUI to, for example, review data for the status and progress of a patient's therapy regimen (e.g., health scores, milestones and achievement indicators, patient-inputted data), review updated data for the patient, and transmit data updates for the patient's data record, among other operations.

Patient Device 109

A patient device 109 may execute any number of software application programs, including, for example, a therapeutic software application program of the digital therapy 101, where the therapeutic software allows the patient to interact with the services and features of the digital therapy 101. The patient device 109 may further exchange data and/or machine-executable instructions with, for example, an operations server 103 via one or more networks 115. It should be appreciated that a patient device 109 may be any computing device comprising a processor and machine-readable memory capable of executing the processes and tasks described herein. Non-limiting examples of a patient device 109 may include a workstation computer, laptop computer, a server, a mobile device (e.g., smartphone, tablet), and the like. In operation, the patient device 109 may receive data and/or machine-executed instructions related to a patient's therapy regimen, and display and capture relevant data via one or more GUIs. A GUI may display directions to a patient so they may follow the patient's therapy regimen.

Consumer Devices 110

Consumer devices 110 may be devices functioning as data sources generating and providing data inputs to the digital therapy 101. Non-limiting examples of a consumer device 110 may include a wearable device 110a (e.g., fitness tracker) and smart device 110b (e.g., smart scale), among other intelligent electronic devices (e.g., electronic body metric measurement devices, electronic sphygmomanometer, electronic blood glucose monitor, electronic cholesterol monitor). A consumer device 110 may generate data containing body measurement data (e.g., weight, A1C, HDL, LDL, blood pressure) in any number of formats and communicate the data to any number of devices of the system 100 (e.g., a patient device 109, operations server 103, coach device 107, provider device 113). The communication may be performed using any number of wired or wireless technologies (e.g., Ethernet, LAN, WI-FI®, BLUETOOTH®).

In some embodiments, a patient device 109 may receive data from one or more consumer devices 110, such as smart home devices, wearable devices, and the like. The patient device 109 may receive data through wired (e.g., USB®, FIREWIRE®, wired LAN) or wireless (e.g., BLUETOOTH®, WI-FI®) connections and then forward the data to the operations server 103. In some cases, the patient device 109 (e.g., smartphone, tablet) may receive and transmit, to and from the operations server 103, the data captured by the consumer device 110 at a given interval (e.g., every few minutes, hourly, daily). In some cases, the patient device 109 may receive and transmit data to the operations server 103 in response to receiving data or instructions from a consumer device 110. The data may then be stored into an operations database 105, and in some instances, transmitted and/or presented to, e.g., a coach device 107 or provider device 113 via a GUI. The updates to the operations database 105 may also trigger an action by the operations server 103.

Provider Device 113

A provider device 113 may be computing device operated by a clinician or other provider (e.g., doctor, pharmacist) to interact with the digital therapy 101 when providing related care to the patient. A provider device 113 may be any computing device comprising a processor and memory, and capable of performing the various tasks and processes described herein. Non-limiting examples of a provider device 113 may include a desktop computer, laptop computer, mobile device (e.g., smartphone, tablet), and the like. A provider device 113 may have software published by the digital therapy 101 locally installed. Additionally or alternatively, the provider device 113 may comprise web-browser software that may access a website of the digital therapy 101, whereby the doctor interacts with the digital therapy 101 via the website.

The provider device 113 may communicate with the operations server 103 and, in some implementations, an EMR server 111 of a third-party entity (e.g., insurance company) via one or more networks 115. Through this connectivity, the provider device 113 may transmit and receive patient data, allowing the doctor to query and update patient data using the digital therapy 101 and/or the similar software-based functions of the EMR server 111 (e.g., native software of the EMR service, website of the EMR service). In some embodiments, the data of the EMR server 111 may be integrated or otherwise accessible to the operations server 103 via one or more APIs. As such, the data updates to the EMR server 111 may be added to a patient's data records that are stored in the operations database 105 of the digital therapy 101.

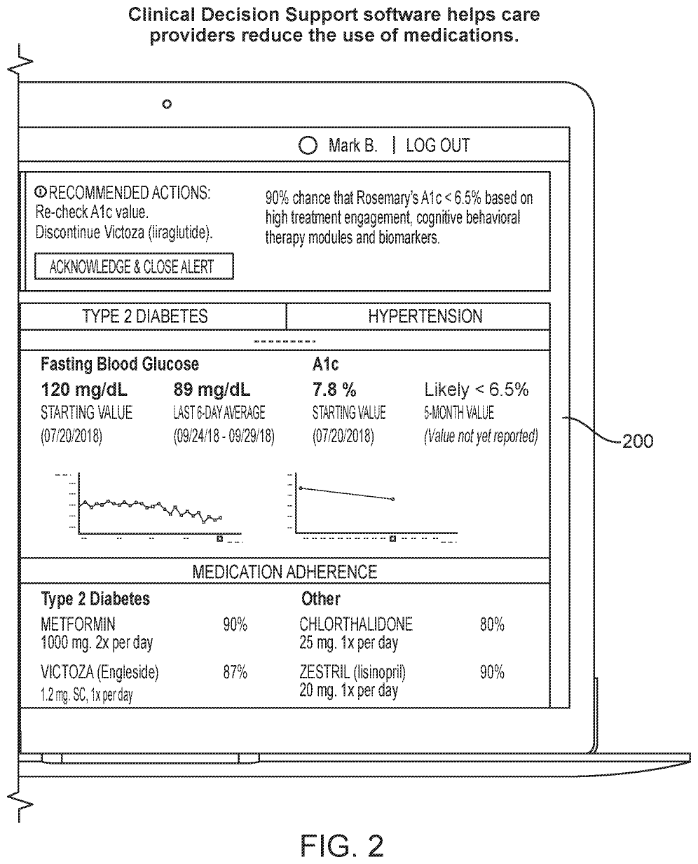

As seen in FIG. 2, the provider device 113 may generate and display a provider GUI 200 to the doctor. The provider GUI 200 may present relevant data and metrics to the doctor based on the information stored in the patient's data records. In operation, the provider GUI 200 may receive the data records and/or provider GUI 200 instructions from the operations server 103 over the one or more networks 115. In some cases, the operations server 103 may determine that a future data score for a particular data field will satisfy a threshold value. In such cases, the operations server 103 may translate an alert to the provider device 113, which may be displayed to the doctor on the provider GUI 200. The alert may be a link to another GUI display or may be a new GUI itself. The provider GUI 200 may display information that provides the reasons the alert was generated and indicate the various data fields and values that drove the alert to be generated.

External Networks 115

As mentioned, the devices of the system 100 may communicate via one or more networks 115. A network 115 may comprise one or more devices that communicate data between devices of the system 100. The components of a network 115 may operate using any number of communications protocols and devices for communicating data among devices, which may include one or more combination of public and/or private networks. In some circumstances, protocols may include telecommunications technology allowing for data and voice data exchange, such as 4G, 3G, LTE, and the like. In such circumstances, the network 115 may comprise telecommunications towers, routers, switches, and trunks. In some circumstances, protocols may include other types of connection-oriented or connectionless data communications protocols, such as Ethernet, TCP/IP, UDP, multiprotocol label switch (MPLS), and the like.

As an example, in FIG. 1, a patient device 109 may be a mobile device (e.g., smartphone) that communicates with an operations server 103 over one or more networks 115. The patient device 109 may communicate (e.g., transmit and receive) data packets with a telecommunications system operated by, e.g., a wireless mobile carrier. Towers, routers, switches, and trunks of the telecommunications system may route the data packets with an internet service provider (ISP) of the digital therapy 101. Routers, switches, and other devices of the ISP may route the data packets to the operations server 103, or other gateway device (e.g., router, firewall), of the digital therapy 101. In this example, a network 115 may comprise the various devices and components used to communicate the data packets between the digital therapy 101 and the patient device 109. One having ordinary skill in the art would appreciate that the devices of the system 100, including the one or more networks 115 of the system 100, may communicate over any number of communication devices and technologies, capable of facilitating networked communication between computing devices, such as, LAN, WAN, InfiniBand, 3G, 4G, or any other computing or digital communication means.

In addition, one having skill in the art would appreciate that devices of the digital therapy 101 may communicate data via one or more internal service-networks (not shown) using any number of networking devices (e.g., routers, switches, trunks, towers) and protocols (e.g., Bluetooth®, TCP/IP, 4G, LTE) capable of transmitting data between devices. In some implementations, internal service-networks of the digital therapy 101 may be private network that are logically and/or physically separated from the components of the one or more networks 115 used by devices that are logically external to the digital therapy 101, such as a patient device 109, an EMR server 111, and provider device 113. For example, an operations server 103 and coach device 107 may be physically located in different geographical locations, but may communicate over public data communications infrastructures of one or more networks 115 (e.g., Internet) using data security measures (e.g., virtual private network (VPN) protocols) to logically separate the internal computing network of the digital therapy 101 from a more public data communications infrastructure.

It should be understood that, throughout this disclosure, stream-oriented connections and/or protocols, including, but not limited to, Quick UDP Internet Connection, UDP Torrent tracker and Stream Control Transmission Protocol may be used in a similar manner as a TCP connection and/or protocol. Additionally, all the protocols running on top of the aforementioned stream-oriented connections and/or protocols, including, but not limited to, HTTP, HTTPS and SPDY, may use features described within the context of the present disclosure by being implemented on top of the applicable stream-oriented connections and/or protocol.

The disclosure is also directed to computer program products comprising software stored on any computer useable medium. Such software, when executed in one or more data processing device, causes a data processing device(s) to operate as described herein. Embodiments of the disclosure employ any computer useable or readable medium, known now or in the future. Examples of computer useable mediums include, but are not limited to, primary storage devices (e.g., any type of random access memory), secondary storage devices (e.g., hard drives, SSDs, floppy disks, tapes, magnetic storage devices, optical storage devices, MEMS, nano-technological storage device, Flash, etc.), and communication mediums (e.g., wired and wireless communications networks, local area networks, wide area networks, intranets, etc.).

It is to be understood that the various embodiments disclosed herein are not mutually exclusive and that a particular implementation may include features or capabilities of multiple embodiments discussed herein.

While the present disclosure refers to packets and/or fields within packets by certain specific names, it is to be understood that these names are not intended to limit the scope of the invention in any way and that any name or designator may be given to packets and/or fields described herein as long as it performs the function and/or serves the purpose described herein. It is also to be understood that the invention is not limited to any particular structure and/or organization of packets and/or fields therein.

While specific embodiments and applications of the present invention have been illustrated and described, it is to be understood that the invention is not limited to the precise configuration and components disclosed herein. The terms, descriptions and figures used herein are set forth by way of illustration only and are not meant as limitations. Various modifications, changes, and variations which will be apparent to those skilled in the art may be made in the arrangement, operation, and details of the apparatuses, methods and systems of the present invention disclosed herein without departing from the spirit and scope of the invention. By way of non-limiting example, it will be understood that the block diagrams included herein are intended to show a selected subset of the components of each apparatus and system, and each pictured apparatus and system may include other components which are not shown on the drawings. Additionally, those with ordinary skill in the art will recognize that certain steps and functionalities described herein may be omitted or re-ordered without detracting from the scope or performance of the embodiments described herein.

The various illustrative logical blocks, modules, circuits, and algorithm steps described in connection with the embodiments disclosed herein may be implemented as electronic hardware, computer software, or combinations of both. To illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application—such as by using any combination of microprocessors, microcontrollers, field programmable gate arrays (FPGAs), application specific integrated circuits (ASICs), and/or System on a Chip (SoC)—but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

The methods disclosed herein comprise one or more steps or actions for achieving the described method, and systems for performing such steps or actions, or a portion thereof. The method steps and/or actions may be interchanged with one another without departing from the scope of the present invention. In other words, unless a specific order of steps or actions is required for proper operation of an embodiment or system, the order and/or use of specific steps and/or actions may be modified without departing from the scope of the present invention.

### EXAMPLES

### Example 1

### Introduction

Modifiable behaviors are responsible for 70% or more of all cardiometabolic diseases. Benjamin et al. Circulation 2019 139:e56-e66; Ford et al. Arch Intern Med 2009; 169:9 Health systems are ill equipped to address the current epidemic of cardiometabolic diseases and, in particular, lack widely available behavioral therapies to address the common root causes of these conditions. Digital therapeutics, software designed to encourage changes in behaviors which treat disease, offer a means to deliver behavioral therapy to large populations and offer potential advantages such as ease of access, ease of use, fewer side-effects and cost-effectiveness compared to pharmacotherapy. Turner et al. JMIR 2018; 20: e207; Berman et al. JMIR Diabetes 2018; 3: e4.

Digital therapeutics also generate readily accessible patient data without requiring an office or lab visit. The data generated are voluminous and vary in both type and quality. These can include remotely sensed measures of physiology (e.g., blood pressure, blood glucose, heart rate variability), behavioral data (e.g., eating, moving, thinking), medication adherence, as well as engagement parameters of additional significance (e.g., app use, geographic location, circadian patterns of use). The best use of these data remains an open question. Feeding data directly into electronic medical records is of limited utility to providers or patients. In contrast, transforming the data into markers of disease status, termed digital biomarkers, could provide clinically actionable insights with or without conventional biometric data. Fritz et al. BMJ Open 2018; 8: e020124. Moreover, digital biomarkers afford a pragmatic approach to remotely monitor patients and intervene on a continuous rather than episodic basis. Greatly expanding opportunities to intervene means that patients have greater access to personalized care, which could improve treatment outcomes.

Machine learning, a type of artificial intelligence (AI) used to make predictions with large and complex datasets, offers a novel approach for creating digital biomarkers. The exponential growth of smartphone use in the United States and advancing interoperability standards allow for digital biomarkers to be compiled across diverse populations and data sources. Machine learning is particularly valuable when there is ambiguity about what variables, or to what extent a set of variables predict an outcome of interest. Such ambiguity is inherent to behavioral interventions, like those used in the treatment of cardiometabolic disease.

Human behavior results from a complex interaction between the anthropogenic features of our living environment, genetic and epigenetic determinants of behavior, neurobiology, social influences and to some degree chance events. Thornton et al. Health Aff 2016; 35: 1416-23; Egger et al. BioMed Res Int 2014; 2014: 1-12. While clinical experience and the scientific literature can identify many variables that influence behavior, the interplay of these variables in a given individual and their environment is difficult for a clinician or patient to discern. This ambiguity limits enthusiasm for behavioral interventions because it makes it difficult for clinicians and patients to rely on behavioral therapy to achieve a predictable level of therapeutic response.

As demonstrated herein, digital biomarkers can reduce ambiguity by predicting current and forecasting future disease status during the course of treatment. Digital biomarkers that serve as markers of current disease status allow for tailoring or adjusting treatment between clinic visits (e.g., when a patient is not doing as well as expected). Similarly, markers of future disease status enable preemptive action, such as adding or subtracting additional treatments, or taking preventive steps to avoid complications of the disease.

In this Example, we present certain digital biomarkers and illustrate their utility in digitally-delivered behavioral interventions to both the patient and prescribing clinician. We describe the analytic process to show that the development of digital biomarkers requires a hypothesis-driven approach, particularly when datasets are small. Finally, using actual examples of digital biomarkers intended to predict blood pressure status, we demonstrate practical outcomes from applying digital biomarkers developed using machine learning.

### Methods

The digital biomarkers discussed herein were generated using data from a digital therapeutic created by Better Therapeutics LLC (San Francisco, Calif.).

The digital therapeutic integrates a mobile medical application (“app”) that delivers behavioral therapy with the support of a remote multidisciplinary care team. The app delivers a personalized behavior change intervention, including tools for goal setting, skill building, self-monitoring, biometric tracking and behavioral feedback designed to provide cognitive training and support the participant's daily efforts to improve overall cardiometabolic disease status. The app facilitates the adoption of evidenced-based behavioral strategies, such as planning and self-monitoring, to increase physical activity and consumption of vegetables, whole grains, fruits, nuts, seeds, beans and other legumes.

Participants were over 18 years of age who self-identified with a diagnosis of hypertension, type 2 diabetes and/or hyperlipidemia. Those reporting to have hypertension at enrollment were offered a free Omron 7 Series Upper Arm Blood Pressure Monitor (Omron Healthcare Inc, Kyoto, Japan) to use during the intervention and to keep at its conclusion. This 12-week intervention was available to all participants at no cost.

This analysis of existing data from participants using the digital therapeutic was approved and overseen by Quorum Review Institutional Review Board and a waiver of informed consent was granted for this retrospective analysis.

The clinical intent of this biomarker exploration was to generate digital biomarkers that could improve treatment outcomes in future participants with hypertension for whom blood pressure was not optimally controlled despite the use of pharmacological treatment. Prior participants that met the following criteria were identified: baseline blood pressure was 1) at or above the cutoff for stage I hypertension (systolic ≥130 or diastolic ≥80) and, 2) recorded no more than 2 weeks prior to or 2 weeks after the start of the intervention. The baseline value was calculated by taking an average of all values reported in a 6-day interval defined as starting with the date of the first blood pressure value reported and all values reported in the following 5 days.

The development of a predictive model using a training dataset requires all participants to have known outcomes. This is referred to as the “ground truth” in machine learning. In this case, the ground truth was change in blood pressure from baseline. The follow-up blood pressure values were calculated by taking an average over a 6-day interval ending with the last blood pressure reported and all values in the previous 5 days. Of the participants identified, we only included those with a follow-up blood pressure value in weeks 7 to 14 of the intervention.

For the purpose of developing biomarkers that predict blood pressure status, we further constrained the training dataset to include participants who used versions of the app which enabled automatic blood pressure tracking.

The intent of machine learning is to train an algorithm that can predict a specific outcome, termed the “response variable.” A suitable response variable can preferably be both clinically relevant and sufficiently, but not necessarily universally, prevalent in the population of interest. For example, if the outcome is “any degree of improvement”, but “any degree of improvement” occurs in >95% of participants in the training dataset, then a predictive model may appear to work well, but could actually be invalid.

We chose response variables that were well distributed in the training dataset, as detailed in the results section, and were also clinically meaningful in the management of hypertension. Specifically, we chose to predict: 1) a systolic blood pressure improvement of 10 mmHg or higher near the end of a 12-week intervention period (defined as 7 to 14 weeks after start), because 10 mmHg has been well demonstrated to be clinically meaningful and that degree of change is near the mean for participants in the training dataset (66/135 participants); and 2) a reduction in blood pressure to the elevated range or below (systolic ≤130), because this level of blood pressure control would signal a clinician to stop adding additional pharmaceuticals or consider reducing or deprescribing pharmaceuticals (48/135 participants). See, e.g. Ettehad et al. Lancet 2016; 387: 957-67

A particularly useful digital biomarker that predicts blood pressure status can compute with data collected in a short period of time, and in less time than typically occurs between clinic visits. This means the biomarker could be used to intervene between office visits and could play a role in addressing clinical inertia that limits primary care providers ability to optimize blood pressure control in their patients. Ogedegbe J. Clin. Hypertens (Greenwich) 2008; 10: 644-46. To demonstrate proof of concept, we chose a 28-day training interval, meaning that we trained machine learning models on the first 28 days of patient data to evaluate whether it could predict blood pressure change in weeks 7 to 14. We hypothesized that data collected within this short training window could sufficiently represent changing behavioral patterns and treatment response, so as to predict future blood pressure status.

There are many valid methodologies for machine learning. These methods can be categorized by the type of learning involved—supervised or unsupervised. A modeling technique appropriate for the size of the dataset and nature of response variables chosen should be selected. For the biomarkers presented herein, we utilized random forest models, which is a form of supervised classification learning that can reduce overfitting in small datasets. Breiman Machine Learning 2001; 45: 5-32. The models are supervised because the ground truth (i.e., actual blood pressure change) for each participant in the training dataset is labeled. The models are attempting to learn whether the classification was or was not achieved; for example, will a participant achieve a >10 mmHg blood pressure reduction, or not?

In addition to classification labels, random forest models must be trained on a set of explanatory variables. For a small training dataset, each model should have a limited number of variables so as to avoid excess noise and overfitting that can lead to reduced generalizability. These explanatory variables are typically selected by hypothesis. For example, we hypothesized that baseline blood pressure and achievement of behavioral goals would influence the degree of blood pressure change observed and used these as explanatory variables. In a large dataset, feature engineering can be used to identify the most predictive explanatory variables.

For each biomarker model, denoted SC (systolic change of 10 mmHg or more) or ER (elevated range achieved), we used 13 explanatory variables, which can be categorized as engagement or biometric variables. Engagement variables are counts of actions related to the use of the digital therapeutic, including count of all meals reported, plant-based meals reported, physical activity reported, and length of exposure to the intervention. Other engagement variables such as skill-module(s) completed are contemplated. Biometric variables included baseline systolic, baseline diastolic, mean systolic and diastolic at training window end, initial systolic and diastolic change (end training mean—baseline), minutes of physical activity, and baseline Body Mass Index (BMI). Additional explanatory variables can include known predictors of treatment response (e.g., time since diagnosis, medication adherence or change).

We trained each biomarker model on data generated during a 28-day training window, starting with participant day 0 (sign up) up to day 28 (a third of the way through the studied 12-week intervention period). We utilized hyperparameter optimization to minimize overfitting and to achieve the maximal leave-one-out cross-validated area under the receiver operating characteristic curve for both models.

Performance of each biomarker model was assessed using leave-one-out cross-validation, which is a common technique for use in samples of this size. Liu et al. PLoS ONE 2014; 9: e84408. This was done by training each model on N−1 samples of the data and then making a prediction on that one sample that was left out, producing an “out of sample’ prediction for all N samples. The N predictions were pooled to generate the classification variables of the receiver operator characteristic curve (ROC), the area under the curve of the ROC (AUROC) and a confusion matrix of true versus predicted values. Airola et al. Comput Stat & Data Anal 2011; 55: 1828-44.

For each biomarker model, the ROC curve illustrates predictive ability of the response variable (in this case systolic change of 10 mmHg or move to a range of elevated BP) at different thresholds of discrimination. At each prediction discrimination threshold, the ROC displays the false positive rate (FPR) against the true positive rate (TPR). The FPR is the ratio of truly negative events categorized as positive (FP) to the total number of actual negative events (N). Specificity or true negative rate of a model is calculated as 1−FPR and is an indication of how well a model does in correctly identifying those who do not achieve a successful outcome, as defined by the response variable.

Since the intended application of these biomarkers is analogous to a diagnostic test, which are traditionally evaluated based on their specificity, we evaluated model performance at a specificity of 90% (FPR=0.10). A low FPR minimizes the number of participants who the model would predict to achieve a healthier state who actually will not. In turn, this minimizes the number of participants who might be erroneously taken off blood pressure medicine as a result of an erroneous prediction. It is less critical to avoid labeling participants who had achieved a healthier state as though they had not. This is why we choose a discrimination threshold with a low FPR, and then evaluate the TPR at that point.

As a further validation step, we examined the performance of each biomarker by excluding the four explanatory variables that capture blood pressure change in the training window. While we hypothesized that these models would perform less well, they serve to test the concept that a digital biomarker that predicts blood pressure status can be generated without using ongoing blood pressure data or without using complete ongoing blood pressure records. These validation models using only app engagement and other biometric variables, are denoted SC-APP and ER-APP.

Digital biomarkers that are generated using machine learning do not need to be viewed as a black box. Instead, explainable AI techniques are available that can provide more granular details about the explanatory variables that influenced the prediction made. Explainable AI can afford both individual participant and population level insights.

We used the Tree Shapley Additive Explanation (SHAP) algorithm on the random forest models to generate more interpretable predictions at the participant level. The SHAP algorithm assigns each explanatory variable an importance value for each prediction. Using SHAP on a machine learning model is analogous to coefficient analysis in classical regression. Similar to coefficient analysis, it can be used to determine the relative importance of explanatory variables in addition to determining which explanatory variables drove a particular prediction. Predictions start at a base value that is the expectation of the response variable. For binary classification models, this is defined by the proportion of outcomes by class (e.g., the proportion of participants who successfully reduced their blood pressure). Then SHAP values attribute to each explanatory variable the change in expected model prediction given the addition of that explanatory variable. This provides insight into how much each explanatory variable positively or negatively impacts the prediction made for each participant. The final prediction probability of whether the participant will achieve the response variable is the sum of the base value and all of the explanatory variable attributions. Lundberg & Lee (arxiv.org/1706.06060) describe methods for feature attribution in tree ensembles, including the application of the SHAP algorithm and calculation and/or attribution of importance values for clustering disease (Alzheimer's) sub-types.

The present inventors have determined that individual SHAP values, and other methods of calculating and/or assigning an importance value) can be used to provide specific behavioral feedback to participants, and thus provide an improved method of motivating a change in behavioral pattern that may improve treatment outcomes. In particular, explanatory variables that are theoretically modifiable (such as minutes of exercise, or number of plant-based meals consumed) can be displayed (e.g., rank ordered by importance value) to motivate changes, whereas fixed explanatory variables (such as baseline values) can be displayed to provide context. SHAP values for all participants can also be plotted to reveal the overall ranking of variables in the population studied. These variable rank lists can then inform hypotheses about how to further improve the design of the digital therapeutic to optimize clinical outcomes.

All machine learning model development was done using open-source packages in Python. The packages include but are not limited to Scikit-Learn, SHAP, Pandas, and Numpy.

### Results

The training dataset contained 135 participants who met the inclusion criteria. The mean age was 54.9 years (95% CI 53.5, 56.3), mean baseline BMI was 34.5 (95% CI 33.1, 35.8) and 83% (112/135) were female. Based on the 2017 ACC/AHA definition, half of the participants (68/135) had stage 1 hypertension at baseline, with the other half (67/135) having stage II hypertension at baseline. Of those with stage 1 hypertension at baseline, 51.5% (35/68) had isolated diastolic hypertension (i.e., diastolic BP 80-90 mmHg). Of those with stage II hypertension at baseline, 14.9% (10/67) had isolated diastolic hypertension (i.e., diastolic BP≥89 mmHg). On average, participants contributed 3 blood pressure readings to the baseline value (95% CI 2.5, 3.4) and 2.5 readings to the end-intervention value (95% CI 2.2, 2.9). Baseline characteristics are listed in Table 1.

TABLE 1
Participant characteristics at baseline

| Participant | Total | | Stage I BP | | Stage II BP | |
|---|---|---|---|---|---|---|
| Characteristics | (n = 135) | | (n = 68) | | (n = 67) | |
| Age (years), | 54.9 | (53.5 to 56.3) | 55.7 | (53.7 to 57.7) | 54.2 | (52.1 to 56.2) |
| mean (95% CI) | | | | | | |
| Body mass index | 34.5 | (33.1 to 35.8) | 33.8 | (31.9 to 35.6) | 35.2 | (33.2 to 37.2) |
| (kg/m²), mean (95% CI) | | | | | | |
| Female, n (%) | 112 | (83) | 54 | (79.4) | 58 | (86.6) |
| Systolic BP (mmHg), | 138.9 | (136.2 to 141.6) | 127.9 | (126.1 to 129.7) | 150.0 | (146.6 to 153.5) |
| mean (95% CI) | | | | | | |
| Diastolic BP (mmHg), | 87.8 | (86.1 to 89.4) | 82.3 | (81.1 to 83.6) | 93.3 | (90.7 to 95.8) |
| mean (95% CI) | | | | | | |
| Isolated diastolic | 45 | (33.3) | 35 | (51.5) | 10 | (14.9) |
| hypertension, n (%) | | | | | | |
| BP medications | | | | | | |
| (count), mean (95% CI) | 1.3 | (1.1 to 1.5) | 1.2 | (1.0 to 1.4) | 1.4 | (1.1 to 1.7) |

Over the intervention period examined, systolic blood pressure changed by −12.7 mmHg (95% CI −14.8, −9.6), diastolic blood pressure changed by −7.1 mmHg (95% CI −9.0, −5.2) and the mean duration of days between baseline and most recent value for blood pressure was 79.3 days (95% CI 76.8, 81.9). Of all participants, 35.6% (48/135) shifted to a blood pressure range below stage I (<130/80). A shift to a normal range was seen in 16.4% (11/67) of those starting with stage II hypertension and 29.4% (20/68) of those starting with stage I hypertension.

The random forest classifier achieved optimal performance with 100 trees and a minimum of 3 samples per leaf node for the SC model. For the ER model optimal performance was achieved with 400 trees and a minimum of 5 samples per leaf nodes.

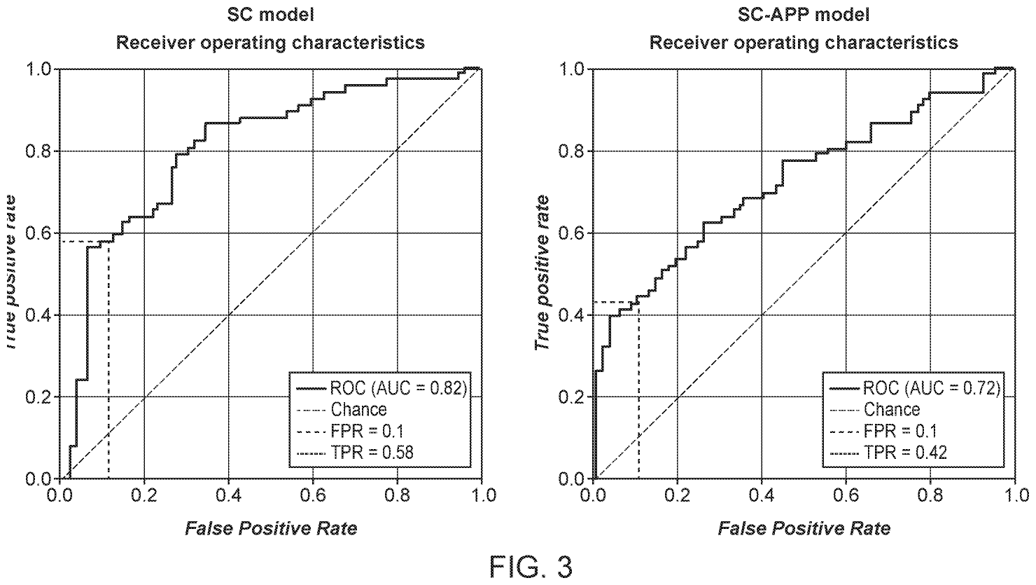

Biomarker models were assessed at the operating point on each ROC that was as close as possible to a FPR of 10%. The SC model (predicting a systolic change of 10 points) was assessed at a FPR of 10%, which means that 10% of participants who didn't achieve a reduction in systolic blood pressure of 10 mmHg were labeled as though they had. Evaluating the model at 10% FPR, we were able to achieve a TPR of 58%. This means that 58% of participants who achieved a reduction in systolic blood pressure of 10 mmHg were labeled correctly. The AUROC was 0.82, model specificity (1−FPR) was 90%, sensitivity (TPR) was 58% and accuracy ((TP+TN)/n) was 74%. In the SC-APP model, where variables related to changes in blood pressure were removed, the AUROC was 0.72 and at a FPR of 10% (specificity of 90%), the TPR was 42%. The resultant receiver operator curves for these 2 models can be seen in FIG. 3.

The biomarker models exploring the ability to predict a shift down to a blood pressure range of elevated or better (ER and ER-APP) also demonstrated predictive capacity, but less so than the SC models. For the ER model, the AUROC was 0.69 and at a FPR of 9% the TPR was 32%. When the BP change variables were removed, in the ER-APP model, the prediction ability was only slightly above chance (AUC=0.53, TPR 26% at FPR of 12%).

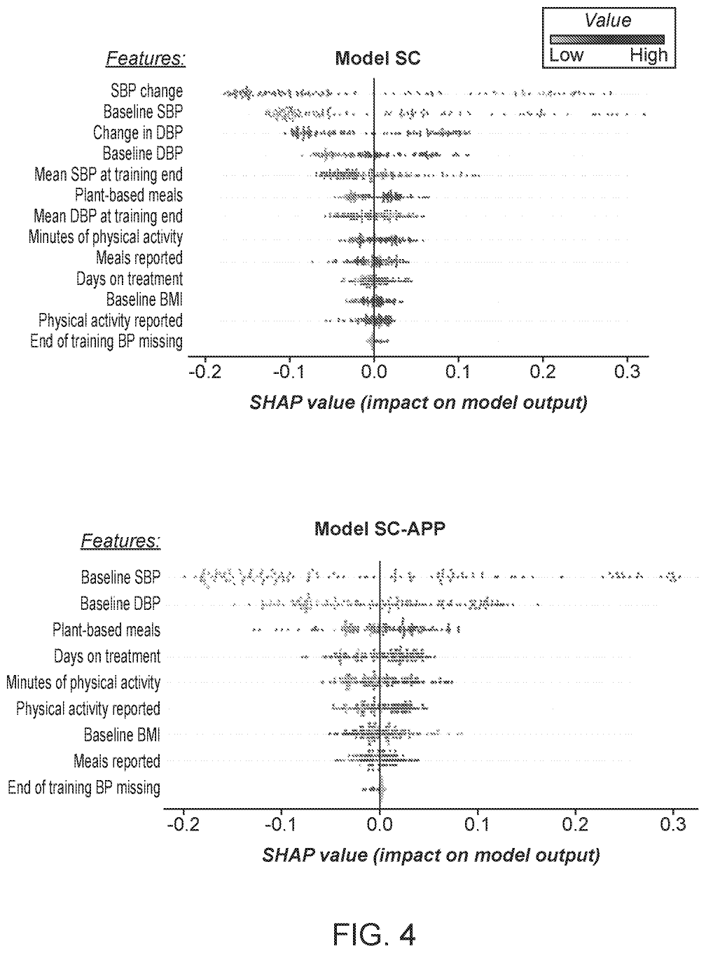

Plots of the Tree SHAP algorithm results for the SC and SC-APP models are shown in FIG. 4. Explanatory variables on the y-axis are ordered from most to least predictive based on their average absolute contribution to the response variable. Each dot represents the SHAP value of that variable for one participant and the placement of the dots on the x-axis indicate if the contribution was subtractive or additive for a specific participant. The color of the dot is indicative of the value for that variable, with highly positive values displayed as red and low or negative values showing up as light blue. The plot for the SC model reveals that explanatory variables related to blood pressure were top contributors to the prediction. For example, the distribution of dots across the x-axis for the first variable listed shows that improvements in systolic BP early in the intervention, as seen by the blue and purple dots to the right of 0 on the x-axis, contributed positively to the prediction that a participant would succeed. Behavioral variables also had predictive power. For example, a high count of physical activity minutes and plant-based meals reported positively contributed to a prediction of success for most participants.

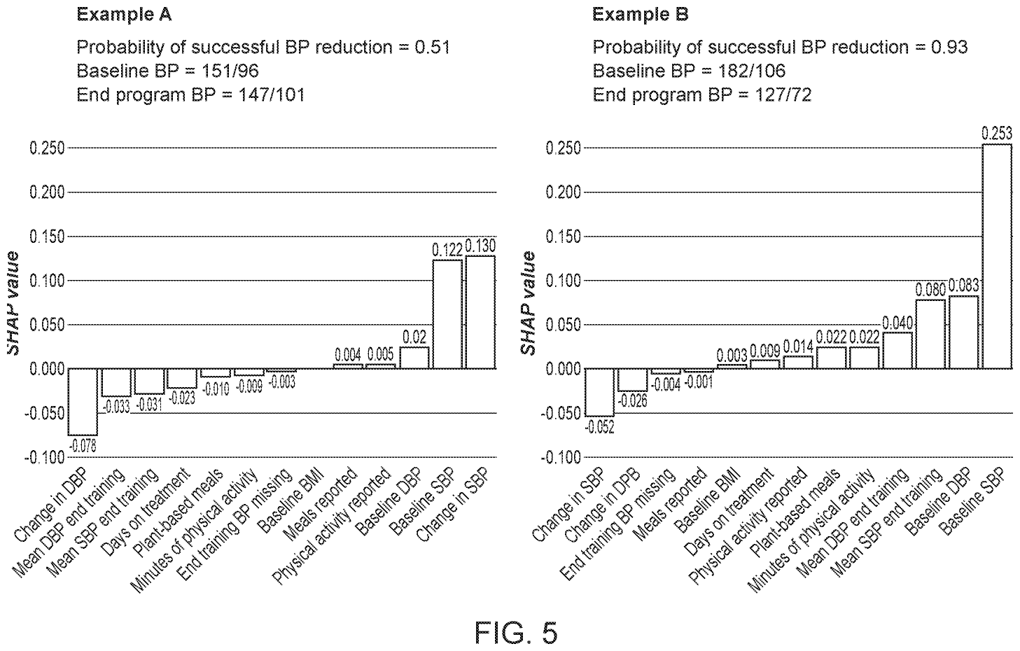

Shapley values can be aggregated and illustrated for every participant. A plot of the SHAP values helps to visualize which variables contributed most to a low or high prediction of success for an individual participant. In FIG. 5, we display the SHAP values for two participants, one with a lower than expected probability of success (example A), and one with a higher than expected probability of success (example B). In example A, the participant experienced a large improvement in their systolic blood pressure in weeks 3 and 4 (−14 mmHg), yet is given a low probability of sustaining this improvement at the end of the intervention period. This surprisingly low probability is explained by the SHAP values, which reveal low counts for several behavioral explanatory variables, such as the number of plant-based meals and minutes of physical activity reported. This data can be automatically translated into a simple explanation to the participant, that their probability for sustaining meaningful change could be higher if they made incremental improvements in their meal and activity pattern. Furthermore, the exact number of additional meals and activity minutes to accrue per week to sufficiently increase the probability of success could be calculated, to motivate the participant and to give meaning to the additional efforts prescribed.

In example B, the participant has evidenced no improvement in systolic blood pressure at the end of week 4, yet they are predicted to meaningfully improve blood pressure by the of the treatment period. This unexpected prediction is explained by the SHAP values, which show that the combined impact of their baseline blood pressure and behavioral explanatory variables suggests a high likelihood of success. This data can be automatically translated to provide timely encouragement to the participant to maintain or advance their behavioral changes even though their blood pressure has not yet responded.

### Discussion

Demonstrated herein are clinically relevant digital biomarkers that were developed using a small training dataset from a digital therapeutic. We demonstrated that 28 days of data can be transformed, using machine learning, into a digital biomarker that predicts the degree of treatment response, in this case whether a meaningful drop in blood pressure will occur at the end of the treatment period.

There are many ways to use such a biomarker in practice to tailor behavioral treatment and improve outcomes. For a patient, these biomarkers can be used as a continuous form of treatment feedback and behavioral reinforcement. The probability of a significant treatment response can be translated into a health score, much like a credit score. Since this score could be recalculated with every new engagement recorded in the digital therapeutic, it would serve to motivate app use and reinforce healing behaviors. In addition, the biomarker output can be made more meaningful using explainable AI techniques. For example, SHAP values can be translated into a prioritized list of behavioral actions to help a patient focus their attention on efforts that are most predictive of success.

For clinicians and health systems, digital biomarkers can function as a form of automated patient monitoring. The probability of a positive treatment response can be translated into a clinical alert by setting an acceptable specificity-sensitivity threshold for each biomarker paired with a duration of time above this threshold. Like any diagnostic test, the performance characteristics of the alert should be made known to those acting upon it. Since such an alert would be intended to influence treatment decisions, for example via a clinical decision support tool, specificity-sensitivity pairs need to be evaluated from a risk-benefit perspective. For instance, how do the risks associated with false positives and false negatives compare to the benefits of identifying true positives and true negatives? To accurately weigh these risks and benefits requires us to understand the context that the biomarker and therapeutics are used. Current clinical practice, for example, is plagued by high rates of clinical inertia (i.e., a lack of timely and appropriate treatment decisions). Therefore, a higher false positive rate may be tolerated as a trade-off for an easier-to-access biomarker.

For a developer of digital therapeutics, digital biomarkers provide not just a way to personalize treatment and communicate clinical status to providers, but also a way to better understand what variables within the therapeutic are most predictive of clinical outcomes. These data can be used to guide the ongoing refinement of a digital therapeutic. When datasets are of sufficient size, the machine learning techniques used to generate digital biomarkers can also be applied to identify distinct digital phenotypes, that is, unique patterns of engagement with a behavioral intervention that represent meaningful subpopulations who share the same diagnosis. Identifying and targeting treatment to previously unknown subpopulations is thought of as meaningful step towards more personalized medicine.

### Example 2

### Introduction

The incidence of type 2 diabetes is at pandemic levels and is continuing to grow in the United States and globally. Despite the increased use of medications and introduction of new pharmacological treatments, current research indicates that glycemic control among those with diabetes is not improving. While type 2 diabetes is currently considered a progressive chronic disease, there is growing evidence that it can be treated and in some cases reversed, with comprehensive lifestyle changes. Behavioral interventions that successfully implement lifestyle changes have potential benefits over traditional therapies including fewer adverse side effects as well as lower healthcare costs and greater overall acceptability.

Lifestyle behavioral interventions have been shown to outperform medications in the treatment of diabetes but there is the persistent challenge of extending access to the larger diabetes population. Digital therapeutics that deliver behavioral interventions have the potential to increase access because they are inherently scalable and can be reached outside of traditional brick-and-mortar constraints. Digital therapeutics are defined as digitally delivered interventions for treating disease and have the advantages of ease of access, ease of use and cost-effectiveness.

Digital therapeutics generate large amounts of accessible patient data, including remotely sensed measures of physiology (e.g blood glucose, blood pressure), behavioral data (e.g. eating, moving) and engagement parameters (e.g. intervention use frequency, counts of features used). The transformation of this data into markers of disease status, termed digital biomarkers, can provide clinically actionable insights with or without traditional biometric data. They can also allow for a pragmatic approach to remotely monitor patients and intervene on a continuous rather than episodic basis.

Machine learning, a type of artificial intelligence used to make predictions with large and complex datasets, offers a novel approach for creating digital biomarkers. This methodology is extremely valuable when there is ambiguity regarding what variables are predictive of an outcome. This ambiguity imposes a challenge for clinicians and patients who rely on lifestyle behavioral interventions to predict a therapeutic response. A machine learning model for predicting response to a digital therapeutic, as described herein, can provide feedback to patients and clinicians to inform adjustments to behaviors and treatment goals. In some cases, the model can be applied early in a treatment cycle to inform adjustments to behaviors and treatment goals and thereby intervene before the disease or condition progresses to an advanced stage.

In the current study, we investigate the efficacy of a digital therapeutic application (app) that delivers behavioral therapy to improve glycemic control in adults with type 2 diabetes. An earlier version of the app under investigation, when paired with health coaching, was found to reduce hemoglobin A1c (HbA1c) by 0.8% after 3 months in a sample of 97 adults with type 2 diabetes. In the current study, we examine the efficacy of a stand-alone app to improve glycemic control in patients with type 2 diabetes. This study also assesses the variability of HbA1c change to refine sample size calculations for a subsequent (e.g., larger, longer, and/or randomized) clinical trial.

This study also includes a group of participants who are assigned to a treatment as usual (TAU) arm. This allows a comparison of HbA1c changes between participants on the behavioral intervention and those assigned to the TAU arm. In addition, the data generated through the use of the behavioral app is used to train and test a machine learning model. This model in turn predicts the probability of a participant improving glycemic control, as measured by a ≥0.4% decrease in HbA1c.

The primary end-point is a reduction of HbA1c in adults with type 2 diabetes using a 3-month digital therapeutic behavioral therapy program. An additional end-point is achievement of larger improvements in fasting blood glucose, weight and HbA1c at 90 days in participants assigned to the behavioral app compared to the participants assigned to the treatment as usual group.

Moreover, the data generated through the use of the digital therapeutic over the first 45 days is used to generate a classification model that predicts which participants will have an improvement in HbA1c by ≥0.4% at Day 90.

### Study Outcomes

### Primary Outcome Measures

Machine learning model classification discrimination, as described by the area under the receiver operating characteristic curve (AUC ROC) at Day 45.

Machine learning model classification calibration, as described by a reliability plot at Day 45.

Average HbA1c change at 90 days in both groups.

### Secondary Outcome Measures

Machine learning model classification performance at the end of each week of treatment, as described by the estimated sensitivity, negative predictive value, positive predictive value at 95% specificity, and the Hosmer-Lemeshow test at alpha of 0.05%.

Mean change in SMBG, blood pressure (systolic and diastolic) at 90 days in participants assigned to the behavioral intervention.

Mean change in body weight & non-fasting lipids in both groups.

Changes in SF-12 in both groups

Adverse events (AEs) in both groups

### Overall Study Design

Potential study participants are identified and recruited through digital and other typical recruitment efforts. Participants are identified and directed to a central web portal that provides more detailed information on the study and an electronic informed consent form. Participants that complete the informed consent form, are directed to a study screening survey to determine their overall study eligibility. Participants who are identified as eligible are then be able to generate an electronic laboratory requisition for their baseline HbA1c measurement. Participants are required to have their blood collected at a local validated facility. Participant having a confirmed baseline HbA1c result of >7%, are randomized to either the behavioral intervention app or the treatment as usual group for the duration of the clinical study.

Participants assigned to the behavioral intervention app are asked to engage with one therapy lesson each week during the study, to report plant-based meals and exercise regularly and report their blood glucose values daily. Typically, these participants are able to complete these activities in approximately 120 minutes each week.

Participants assigned to the treatment as usual group are asked to continue their usual treatment for the duration of the study. They are asked to complete a health status form every 15 days where they report on any changes in their overall health. Typically these participants are able to complete this activity in approximately 10 minutes every other week.

The overall study duration for both groups is 90 days. At the conclusion of the 90-day study participation, the behavioral intervention group is given the opportunity to continue on the app for an additional 90 days. Similarly, at the end of their 90-day participation, the treatment as usual group is given the option of accessing the behavioral intervention for 90 days. All participants who continue on the behavioral intervention app are asked to take and/or provide an HbA1c value at Day 180.

Additional data collected includes one or more, or all, of the following: height, weight, concomitant medications, completed health status form, completed SF-12 survey, net promoter score, blood glucose, blood lipids, hemoglobin, and blood pressure. In some cases, blood pressure and/or blood glucose are independently monitored daily or weekly. In cases where blood glucose and/or blood pressure are subject-specific data inputs, the present inventors have determined that the methods described herein are robust to incomplete data. Thus, in some cases, the blood glucose and/blood pressure are, independently, daily or weekly subject-specific data inputs with one, two, three, four or more missing data entries.

### Behavioral Intervention App

The behavioral intervention app used in this study delivers treatment to participants, with type 2 diabetes, using behavioral therapy (BT) which targets behaviors related to achieving glycemic control, and reduces HbA1c. The app functions via participants' smartphone and is downloaded from the phone's corresponding app store. The app delivers the BT intervention to all participants in the behavioral intervention arm.

The BT process involves: 1) identifying and measuring maladaptive thoughts based on misinformed or false underlying core beliefs (e.g., those related to macronutrient fears, the hedonic nature of eating, physical exertion, other perceived barriers to changing lifestyle) that lead to disease-promoting behaviors; 2) replacing these maladaptive core beliefs and thought patterns with adaptive ways of thinking developed from rational reflection; 3) providing collaborative (between participant and device) construction of behavioral exercises to test core beliefs; and 4) using additional validated behavioral techniques to enhance a participant's capacity to solve problems, plan behaviors, and cope with interfering emotions or thoughts.

The behavioral intervention app asks participants to answer behavioral intake questions (behavioral assessment). During this behavioral assessment, participants are asked to assess the presence and strength of their beliefs and perceived barriers to achieving diet and exercise patterns that are sufficient to improve blood glucose control. This behavioral assessment identifies participant's unconscious beliefs that may be responsible for poor behavioral habits or represent barriers to adopting new helpful habits that influence blood glucose control. Participants are asked to complete this assessment every 4 weeks (3 times during the intervention period and 3 times in the follow-up period) to track changes in their beliefs and to reinforce a primary aim of the behavioral therapy: the self-examination of diabetes-related beliefs.

The behavioral invention app helps participants understand the steps they should prioritize by presenting them with a treatment plan that summarizes their daily and weekly goals. In some cases, one or more of the daily or weekly goals are rank ordered by an assigned importance value. In some cases, the assigned importance value is calculated by feature attribution from a tree ensemble. In some cases, the assigned importance value is a Shapley Additive explanation value, e.g., calculated using a Tree Shapley algorithm. Each week, the app asks participants to complete a new behavioral module, along with one or more skill-based exercises that are related to that particular week's module.

All behavioral intervention participants are exposed to the same course of modules. The modules are short exercises, expected to take between 10-20 minutes to complete. The modules are called “therapy lessons” within the invention app. Each therapy lesson addresses core beliefs in one of the following areas:

Personal beliefs and barriers, such as those related to a participant's ability to change and control his or her behaviors;

Beliefs about macronutrients and the importance of various food types;

Hedonic-related beliefs about pleasant or unpleasant sensations experienced by eating or exercising; and

Beliefs about exercise.

The skill exercises are designed to improve dietary, exercise or supportive behavioral patterns. The method by which participants must practice these skills is designed to enhance executive function tasks such as planning, problem solving and goal setting. Each therapy lesson explains the rationale and benefits of the skill exercise in reference to the core topic being explored.

In addition to completing a therapy lesson and one or more skill exercises, the app asks patients to self-report diet and exercise behaviors, medication adherence, and biometrics (e.g., fasting blood glucose, weight, and, if the patient also has hypertension, blood pressure) each day. These inputs provide important data for the predictive model also under investigation in this study and serve as behavioral prompts for patients to take action to improve their glycemic control and health. Weekly behavioral goals, including diet and exercise behaviors, are determined through a goal setting exercise, and are advanced in a manner most likely to maintain or increase self-efficacy. When self-efficacy is high, the participant has a high probability of achieving his or her goals. Additionally, or alternatively, one or more behavioral goals are rank ordered to prioritize those goals calculated to achieve the greatest improvement in outcome (e.g., by attributing an importance value to one or more behavioral goals).

Participants are asked to engage with one therapy lesson each week of the study, to report plant-based meals and exercise regularly and to use their glucometer daily. It is estimated that participants are able to complete these activities in the app in about 60-90 minutes spread over each study week.

### Treatment as Usual (TAU) Participant Study App

During their participation, TAU participants are prompted to complete a health status survey every 15 days. Additionally, TAU participants are asked to complete an SF-12 survey on Day 14 & Day 75. It is estimated that participants are able to complete these activities in the app in about 10-15 minutes every other week.

### SF-12 Survey

All study participants are asked to complete the SF-12 at Day 14 of their respective program and then again at Day 75. The SF-12 Health Survey is a shorter version of the SF-36 Health Survey that uses just 12 questions to measure functional health and well-being from the participant's point of view. The SF-12 is a validated measure and is a widely used tool for monitoring health, comparing and analyzing disease burden.

### Net Promoter Score (NPS)

All behavioral app participants are asked to complete a net promoter score (NPS) at Day 75 of their program. The NPS is a standardized tool for measuring a participant's overall satisfaction with the program. Participants are asked how likely they are to recommend the app to a friend with a relevant condition and are asked to enter a number from 1 to 10.

### Blood Glucose Monitoring

All behavioral app participants are required to use an FDA approved glucometer during the course of their participation in this study. If they do not currently own an FDA approved glucometer, one is provided to them at no cost.

If needed, meters are shipped by a third party to participants at a mailing address they provide. The meter is shipped with printed instructions and an instructional video is available in the apps to help participants get started. If participants have questions or problems with the meters, the help center within the apps is available.

Participants are asked to use the blood glucose meter once per day over the 90-day study period. Each test requires a blood volume of approximately 0.7 uL and it is estimated participants collect up to a maximum of 63 ul over 90 days.

### Hemoglobin Measurements

A confirmed baseline HbA1c value (e.g., measurement date <30 days before enrollment and/or value >7%) is required for a participant to be randomized into the study. Participants are also be asked to complete an HbA1c test at Day 90. Participants are provided a message in their respective app on Day 75 (e.g., via a push notification) and are then provided with a lab requisition from the same lab as their baseline HbA1c test. HbA1c values collected during the course of the study are shared with the participant and the participant's primary care provider. The measurement of HbA1c at 90-day intervals is considered standard of care in the treatment of type 2 diabetes.

### Blood Lipid Measurements

Participants are asked for a blood sample to test their blood lipid levels at screening as well as Day 90. The lipid panel measures overall cholesterol, triglycerides, high-density lipoprotein (HDL) and low-density lipoprotein (LDL). Active monitoring of lipids is recommended for all adults with diabetes.

### Biometric Alerts

Behavioral app participants self-report biometric values (blood glucose, blood pressure, height, weight in the behavioral intervention app and blood glucose throughout the study. These values are monitored in three ways: 1) app data validation 2) automated participant alerts and 3) communications to the principal investigator and primary care physician.

Values manually entered by participants are flagged in real-time if they are out of the biologically likely range. A message within the app can ask the participant to check the value and correct it as needed is shown before the value is saved.

Saved values that are out of the normal clinical range can generate an automated alert within the app, e.g., and sent, via push notification, to the user. A message can be displayed within the app to the participant that explains the nature of the concern and provides simple steps the participant can take to moderate the risk. Alerts can be generated (e.g., and transmitted to the participant and/or clinician or other provider) for values for blood sugar, blood pressure, HbA1c, blood triglycerides and rapid changes in weight.

In some embodiments, the automated alerts generated by the app are summarized by the unique participant identifier (ID) and shared via a HIPAA compliant web portal to the study's principal investigator. When required, the principal investigator matches the participant ID to the participant's PCP contact information collected in the screening process and then initiates direct communication with the participants' PCP. Primary treatment discretion typically remains with the participant's PCP during the course of the study.

### Health Status Form

All study participants will be asked for self-reported details on changes to their health every 15 days. Participant responses will be actively reviewed by the safety monitoring team as well as the principal investigator. Based upon participant responses, direct follow-up may be required by the principal investigator.

### Statistics

Sample-size calculations are performed for the two primary outcomes: 1) mean change in HbA1c 2) performance of a binary classification model for predicting HbA1c at day 90.

The primary outcome of mean change in HbA1c is powered to provide treatment size estimates for a subsequent larger & longer randomized clinical trial of this intervention. Estimating an overall 30% attrition rate during the study, approximately 450 participants are enrolled to have a mean change for HbA1c at the 90-day endpoint in 315 participants. This sample size assumes a 1-tail alpha of 0.05, with approximately 80% power.

The size estimate for the second primary outcome of performance of a model to predict a 90-day improvement in HbA1c≥0.4% uses a method for determining sample size needed for a training data set developed by Figueroa et. al. BMC Med Inform Decis Mak 2012 Feb. 15; 12:8. The method uses a small training data set to compute points along the learning curve. These points are used to fit a power law model of performance as a function of sample size. This model can then be used to forecast performance, and confidence intervals on performance, at larger sample sizes. Using 139 samples collected as part of an initial trial, we used the above technique to determine that sample size of 210 study completers is needed to develop a model with an area under the receiver operator characteristic curve (AUC ROC) of 0.79 (95% CI 0.76 to 0.82). Accounting for approximately 30% attrition rate, approximately 300 participants are enrolled to meet this outcome.

SAS (Statistical Analysis System) is used to perform the descriptive statistical analysis. Parametric methods such as paired Student's t-test are used to assess the change over time in continuous variables; and non-parametric methods such a Chi-Square are used to assess change in binary or other discontinuous variables. Multivariable methods are appended as warranted to adjust for potential confounding factors.

The predictive model determines the probability that a participant achieves a clinically meaningful improvement in HbA1c (≥0.4%) by the end of the treatment period (Day 90). The inputs to the model include one or more, or all, of the following:
a. Participant profile data at baseline (e.g., demographic variables, baseline HbA1c, body mass index, duration of diabetes); and
b. Data acquired during use of the apps (e.g., therapy lessons completed and skills practiced; responses to assessment questions within each therapy lesson; fasting blood glucose values).

The output of the algorithm is the percent probability that the participant achieves a 0.4% or more reduction in HbA1c by the end of the treatment period. Analysis is done to calculate the sensitivity and specificity of the model results. These results are used to inform refinement of the explanatory features included in the predictive model. In some cases, an importance value for one or more, or all, of the explanatory features is calculated (e.g., by calculating a SHAP value).

Performance the predictive model is assessed in regard to calibration and discrimination. Calibration of the predictive model is assessed using the decile-based Hosmer-Lemeshow goodness-of-fit-test plot (reliability curve or calibration plot). The plot is characterized by the distance between the calibration slope (45° line) and the values for the midpoint of each decile on the x-axis and the observed rate on the y-axis. Well calibrated predictions fall closely to the calibration slope. In addition, other tests of calibration are assessed, including sensitivity (true positive rate) and specificity (true negative rate), positive predictive value and negative predictive value.

Analysis of discrimination evaluates how well the model does in differentiating those participants more likely to have a ≥0.4% improvement in their HbA1c at 90 days from those who are not. This is assessed using leave-one-out cross-validation. This method trains the model on N−1 samples of the data and then generates a prediction on that one sample that was left out, producing an “out of sample’ prediction for all N samples. The N predictions are then pooled to generate the classification variables of the receiver operator characteristic curve (ROC), the area under the curve of the ROC (AUROC) and a confusion matrix of true versus predicted values. In some embodiments, an AUC of 0.65-0.75 is considered “useful” or “possibly useful”, an AUC >0.75 is considered “clearly useful.”

All patent and non-patent references cited in the present specification are hereby incorporated by reference in their entirety and for all purposes.

1. A computer-implemented method for managing chronic medication in a patient having a health disorder, said method comprising:
a. providing to said patient a digital therapy for achieving one or more therapeutic milestones for said disorder;
b. collecting subject-specific data values associated with a medication adjustment threshold for said disorder;
c. determining by way of predictive analytics using said subject-specific data values whether a medication adjustment threshold has been or will be reached; and
d. providing to the clinician or other provider for said patient a medication adjustment alert and/or recommendation if said threshold has been or will be reached within a treatment period.

2. The method according to claim 1, wherein said health disorder is a cardiometabolic disorder.

3. A computer-implemented method for treating a patient having or at risk of having a cardiometabolic disorder, said method comprising:
a. providing to said patient a digital therapy for achieving one or more therapeutic milestones for said cardiometabolic disorder;
b. collecting subject-specific data values associated with a medication adjustment threshold for said cardiometabolic disorder;
c. determining by way of predictive analytics using said subject-specific data values whether a medication adjustment threshold has been or will be reached; and
d. providing to the clinician or other provider for said patient a medication adjustment alert and/or recommendation if said threshold has been or will be reached within a treatment period.

4. The method according to any one of claims 1 to 3, wherein said subject-specific data values comprise one or more engagement subject specific values and one or more biometric subject-specific values.

5. The method according to claim 4, wherein said subject-specific data values comprise biometric measurements (e.g., how often measured and what change from baseline), medication adherence, descriptive or demographic data (e.g., location, gender, email service used, consumer activities), service-interaction data (e.g., number of interactions with a coach) and software-interaction data (e.g., actions or tasks performed, software features accessed by a patient, type of data captured from patient device, amount and/or frequency of interactions with software features), and the like.

6. The method according to any one of claims 1 to 4, wherein said determining step comprises a multi-factorial weighted analysis and/or machine learning of two or more, three or more, four or more, five or more, six or more, seven or more, eight or more, or all of the subject-specific data values collected from the patient, performed by an operations server.

7. The method according to any one of claims 1 to 5, wherein said treatment period is at least about one, two, three, four, five, six, seven, eight, nine, ten, eleven, twelve, thirteen, fourteen, fifteen or sixteen weeks.

8. A system for managing a therapy regimen, the system comprising:
an operations database comprising non-transitory machine-readable data configured to store one or more patient records;
an operations server comprising a processor configured to:
generate a first algorithm for determining a therapy regimen for a patient based upon one or more data fields of a plurality of patient records in the operations database;
generate a second algorithm for determining a likelihood of a value change based upon the one or more of the plurality of patient records;
generate the therapy regimen for a patient based upon the one or more data fields of the patient record according to the first algorithm;
determine the likelihood of the value change based upon the one or more data fields of the patient record according to the second algorithm;
update a medication-value of the therapy regimen, in response to determining that the likelihood of the value change satisfies a medication-update threshold; and
transmit, to a GUI of a provider device, at least one data field, the medication-value, and one or more fields of the therapy regimen.

9. The system of claim 6, wherein the operations server is further configured to:
receive patient data from a patient device;
store the patient data into the patient record of the patient; and
transmit at least one data field to a GUI of the patient device.

10. The system of claim 7, wherein the processor is further configured to receive patient data from one or more devices over one or more networks, and store the patient data into the one or more data fields of the one or more patient records; and
wherein the processor uses at least one data field of the patient data for the second algorithm.

11. The system of claim 8, wherein the operations database is further configured to generate the patient record in the operations database using the patient data, and wherein the patient data and the patient record are each associated with the patient.

12. The system of claim 8, wherein the processor is further configured to update the second algorithm based upon updated values of the one or more data fields received from at least one device over the one or more networks.

13. The system of claim 6, wherein the operations server is further configured to communicate patient data with an EMR server over one or more networks.

14. The system of claim 6, wherein the operations server is further configured to communicate patient data with one or more consumer devices, each consumer device configured to generate patient data that is stored into the patent record of the operations database.

15. A computer-implemented method for predicting a health outcome in a patient having a health disorder, said method comprising:
a. providing to said patient a digital therapy for achieving one or more therapeutic milestones for said disorder;
b. collecting subject-specific data values associated with said one or more therapeutic milestones for said disorder;
c. calculating a health score by way of a classifier system, wherein the classifier system comprises a machine-learning trained biomarker model and the classifier uses said subject-specific data values as input and determines a health score indicating whether a health outcome threshold has been or will be reached as output; and
d. calculating and assigning an importance value for one or more, or all, subject specific data values, for the biomarker model.

16. The computer-implemented method of claim 15, further comprising step e) transmitting to the patient and/or their clinician or other provider one or more of i) the health score, ii) behavioral actions that are predictive of success in achieving the health outcome, and iii) clinical alerts.

17. The computer-implemented method of claim 16, wherein the health score is recalculated with every new engagement entered in the digital therapy.

18. The computer-implemented method of claim 16, wherein the one or more behavioral actions are associated with one or more, or all, subject-specific data values rank ordered by the calculated importance value.

19. The computer-implemented method of any one of claims 15-18, wherein said health disorder is a cardiometabolic disorder.

20. The computer-implemented method of any one of claims 15-18, wherein said health disorder is hypertension, or stage I hypertension.

21. The computer-implemented method of any one of claims 15-18, wherein said biomarker model is a machine learning model, preferably a tree ensemble method, more preferably a random forest model.

22. The computer-implemented method of any one of claims 15-21, wherein said biomarker model is trained on one or more engagement and/or biometric subject-specific data values.

23. The computer-implemented method of claim 18, wherein said biomarker model is trained on both engagement subject-specific data values and biometric subject-specific data values.

24. The computer-implemented method of claim 22 or 23, wherein said biomarker model is trained on one or more, or all, of the following subject-specific data values: counts of actions related to the use of the digital therapeutic, count of all meals reported, plant-based meals reported, physical activity reported, length of exposure to the intervention, baseline systolic, baseline diastolic, mean systolic and diastolic at training window end, initial systolic and diastolic change (end training mean—baseline), minutes of physical activity, and baseline Body Mass Index (BMI).

25. The computer-implemented method of any one of claims 15-24, wherein the method comprises calculating the importance value by using gradient values provided by the classifier.

26. The computer-implemented method of claim 25, wherein the method comprises calculating the importance value by accessing information about nodes in a tree model of the classifier.

27. The computer-implemented method of claim 26, wherein the method comprises calculating the importance value by generating a Tree Shaply Additive Explanation value or by performing a regression analysis.

28. The computer-implemented method of any one of claims 15-27, wherein the calculating and assigning the importance value comprises transmitting at least one score request to the classifier to generate the importance value and receiving the importance value from the classifier as a response to each score request.

29. A computer-implemented method of improving a health outcome in a patient suffering from, or at risk of, a cardiometabolic disorder, said method comprising:
a. providing to said patient a digital therapy for achieving one or more therapeutic milestones for said disorder;
b. collecting subject-specific data values associated with said one or more therapeutic milestones for said disorder, including at least one baseline physiologic parameter associated with said health disorder;
c. applying each of the one or more subject-specific data values against a trained classifier, wherein the classifier has been trained with a statistically significant plurality of subject-specific data values associated with one or more of a plurality of subjects, at least some of said plurality of subjects having the cardiometabolic disorder, and
d. based on the applying, calculating a predicted change in the at least one physiological parameter of the patient.

30. The method of claim 29, wherein the method further comprises step e) transmitting the predicted change in the at least one physiological parameters of the patient to the patient and/or a patient's clinician or other provider.

31. The method of claim 30, wherein the cardiometabolic disorder is selected from diabetes, dyslipidemia, obesity, or hypertension.

32. The method of claim 31, wherein the cardiometabolic disorder is diabetes and the at least one physiological parameter is HbA1c level.

33. The method of claim 32, wherein the method further comprises step e) transmitting a predicted change in HbA1c level to the patient and/or a patient's clinician or other provider.

34. The method of any one of claims 31 to 33, wherein the patient is being treated with one or more of sulfonylureas, meglitinides, biguanides, thiazolidinediones, or alpha-glucosidase inhibitors and/or at least some of the plurality of subjects were undergoing treatment with one or more of sulfonylureas, meglitinides, biguanides, thiazolidinediones, or alpha-glucosidase inhibitors.

35. The method of claim 29, wherein the cardiometabolic disorder is dyslipidemia and the at least one physiological parameter is cholesterol level, LDL, and/or HDL.

36. The method of claim 35, wherein the method further comprises step e) transmitting the predicted change in cholesterol level, LDL, and/or HDL to the patient and/or a patient's clinician or other provider.

37. The method of claim 35 or 36, wherein the patient is being treated with one or more of statins, bile acid binding resins, fibrates, niacin, omega-3 fatty acids, cholesterol absorption inhibitors and/or at least some of the plurality of subjects were undergoing treatment with one or more of statins, bile acid binding resins, fibrates, niacin, omega-3 fatty acids, cholesterol absorption inhibitors.

38. The method of claim 29, wherein the cardiometabolic disorder is hypertension and the at least one physiological parameter is blood pressure.

39. The method of claim 38, wherein the method comprises transmitting the predicted change in blood pressure to the patient and/or a patient's clinician.

40. The method of claim 38 or 39, wherein the patient is being treated with one or more of diuretics, beta-blockers, ace-inhibitors, angiotensin II receptor blockers, calcium channel blockers, alpha-2 receptor agonists, alpha-beta blockers, central agonists, adrenergic inhibitors, or vasodilators and/or at least some of the plurality of subjects were undergoing treatment with one or more diuretics, beta-blockers, ace-inhibitors, angiotensin II receptor blockers, calcium channel blockers, alpha-2 receptor agonists, alpha-beta blockers, central agonists, adrenergic inhibitors, or vasodilators.
